(19) United States
(12) Patent Application Publication
(10) Pub. No.: US 2021/0330612 A1
(43) Pub. Date: Oct. 28, 2021

(54) METHODS FOR TREATING CANCER RESISTANT TO CDK4/6 INHIBITORS

(71) Applicant: Radius Pharmaceuticals, Inc., Boston, MA (US)

(72) Inventors: Hitisha PATEL, Malden, MA (US); Teeru BIHANI, Boston, MA (US); Heike ARLT, Arlington, MA (US); Nianjun TAO, Medford, MA (US)

(21) Appl. No.: 17/299,948

(22) PCT Filed: Dec. 6, 2019
(86) PCT No.: PCT/US2019/065005

§ 371 Date: Jun. 4, 2021

Related U.S. Application Data
(60) Provisional application No. 62/776,323, filed on Dec. 6, 2018.

(51) Int. Cl. A61K 31/137; A61P 35/00; C12Q 1/6886

(52) U.S. Cl. CPC A61K 31/137; A61P 35/00; C12Q 2600/158; C12Q 2600/106; C12Q 1/6886

(57) ABSTRACT

Disclosed herein are methods of treating a CDK4/6 inhibitor resistant estrogen receptor alpha-positive cancer in a subject having either a wild-type estrogen receptor alpha and/or a mutant estrogen receptor alpha, the method comprising administering to the subject a therapeutically effective amount of elacestrant, or a pharmaceutically acceptable salt or solvate thereof, wherein the mutant estrogen receptor alpha comprises one or more mutations selected from the group consisting of D538G, Y537X₁, L536X₂, P535H, V534E, S463P, V392I, E380Q and combinations thereof, wherein: X₁ is S, N, or C; and X₂ is R or Q. In some embodiments, the CDK4/6 inhibitor resistant estrogen receptor alpha-positive cancer is selected from the group consisting of breast cancer, uterine cancer, ovarian cancer, and pituitary cancer.

### CROSS REFERENCE TO RELATED APPLICATIONS

This application claims priority under 35 U.S.C. § 119(e) to U.S. Provisional Patent Application No. 62/776,323, filed Dec. 6, 2018. The entire contents of the aforementioned application are hereby incorporated by reference in its entirety, including drawings.

### TECHNICAL FIELD

The present disclosure provides methods of providing anti-tumor activity using elacestrant in cancer models harboring ESR1 mutations resistant to CDK4/6 inhibitors. The present disclosure also relates to methods of treating estrogen positive (ER+) cancers having ESR1 mutations that may contribute to CDK4/6 inhibitor resistance where the cancer is treated using elacestrant.

### BACKGROUND

Breast cancer is divided into three subtypes based on expression of three receptors: estrogen receptor (ER), progesterone receptor (PR), and human epidermal growth factor receptor-2 (Her2). Overexpression of ERs is found in many breast cancer patients. ER-positive (ER+) breast cancers comprise two-thirds of all breast cancers. Other than breast cancer, estrogen and ERs are associated with, for example, ovarian cancer, colon cancer, prostate cancer and endometrial cancer.

ERs can be activated by estrogen and translocate into the nucleus to bind to DNA, thereby regulating the activity of various genes. See, e.g., Marino et al., “Estrogen Signaling Multiple Pathways to Impact Gene Transcription,” Curr. Genomics 7(8): 497-508 (2006); and Heldring et al., “Estrogen Receptors: How Do They Signal and What Are Their Targets,” Physiol. Rev. 87(3): 905-931 (2007).

Agents that inhibit estrogen production, such as aromatase inhibitors (AIs, e.g., letrozole, anastrozole and exemestane), or those that directly block ER activity, such as selective estrogen receptor modulators (SERMs, e.g., tamoxifen, toremifene, droloxifene, idoxifene, raloxifene, lasofoxifene, arzoxifene, miproxifene, levormeloxifene, and EM-652 (SCH 57068)) and selective estrogen receptor degraders (SERDs, e.g., fulvestrant, TAS-108 (SR16234), ZK191703, RU58668, GDC-0810 (ARN-810), GW5638/DPC974, SRN-927, ICI182780 and AZD9496), have been used previously or are being developed in the treatment of ER-positive breast cancers.

SERMs and AIs are often used as a first-line adjuvant systemic therapy for ER-positive breast cancer. AIs suppress estrogen production in peripheral tissues by blocking the activity of aromatase, which turns androgen into estrogen in the body. However, AIs cannot stop the ovaries from making estrogen. Thus, AIs are mainly used to treat post-menopausal women. Furthermore, as AIs are more effective than the SERM tamoxifen with fewer serious side effects, AIs may also be used to treat pre-menopausal women with their ovarian function suppressed. See, e.g., Francis et al., “Adjuvant Ovarian Suppression in Premenopausal Breast Cancer,” the N. Engl. J. Med., 372:436-446 (2015).

Resistance to endocrine therapy is a challenging aspect in the management of patients with estrogen receptor positive (ER+) breast cancer. Recent studies have demonstrated that acquired resistance can develop after treatment with aromatase inhibitors through the emergence of mutations in the estrogen receptor 1 (ESR1) gene. Another mechanism associated with de novo and acquired resistance is the adaptive upregulation of parallel growth-factor signaling pathways as well as crosstalk between these pathways, including those that promote expression of cyclin D1 and activation of Cyclin Dependent Kinase 4 (CDK4) and CDK6 (CDK4/6). While initial treatment with these agents may be successful, many patients eventually relapse with drug-resistant breast cancers. Mutations affecting the ER have emerged as one potential mechanism for the development of this resistance. See, e.g., Robinson et al., “Activating ESR1 mutations in hormone-resistant metastatic breast cancer,” Nat Genet. 45:1446-51 (2013). Mutations in the ligand-binding domain (LBD) of ER are found in 20-40% of metastatic ER-positive breast tumor samples from patients who received at least one line of endocrine treatment. Jeselsohn, et al., “ESR1 mutations—a mechanism for acquired endocrine resistance in breast cancer,” Nat. Rev. Clin. Oncol., 12:573-83 (2015).

Therefore, there remains a need for more durable and effective ER-targeted therapies to overcome some of the challenges associated with current endocrine therapies and to combat the development of CDK4/6 resistance.

### SUMMARY OF THE INVENTION

In one aspect, the disclosure relates to a method of inhibiting and degrading a CDK4/6 inhibitor resistant estrogen receptor alpha positive cancer in a subject comprising administering to the subject a therapeutically effective amount of elacestrant, or a pharmaceutically acceptable salt or solvate thereof.

Embodiments of this aspect of the invention may include one or more of the following optional features. In some embodiments, the CDK4/6 inhibitor resistant estrogen receptor alpha positive cancer is resistant to palbociclib, ribociclib, abemaciclib, or a combination thereof. In some embodiments, the CDK4/6 inhibitor resistant estrogen receptor alpha positive cancer is resistant to palbociclib. In some embodiments, the CDK4/6 inhibitor resistant estrogen receptor alpha positive cancer is resistant to ribociclib. In some embodiments, the CDK4/6 inhibitor resistant estrogen receptor alpha positive cancer is resistant to abemaciclib. In some embodiments, the CDK4/6 inhibitor resistant estrogen receptor alpha positive cancer comprises one or more mutations selected from the group consisting of D538G, Y537X₁, L536X₂, P535H, V534E, S463P, V392I, E380Q and combinations thereof, wherein: X₁ is S, N, or C; and X₂ is R or Q. In some embodiments, the mutation is Y537S. In some embodiments, the mutation is D538G. In some embodiments, the CDK4/6 inhibitor resistant estrogen receptor alpha positive cancer is resistant to a drug selected from the group consisting of anti-estrogens, aromatase inhibitors, and combinations thereof. In some embodiments, the CDK4/6 inhibitor resistant estrogen receptor alpha positive cancer is selected from the group consisting of breast cancer, uterine cancer, ovarian cancer, and pituitary cancer. In some embodiments, the CDK4/6 inhibitor resistant estrogen receptor alpha positive cancer is advanced or metastatic breast cancer. In some embodiments, the CDK4/6 inhibitor resistant estrogen receptor alpha positive cancer is breast cancer. In some embodiments, the subject is a post-menopausal woman. In some embodiments, the subject is a pre-menopausal woman. In some embodiments, the subject is a post-menopausal woman who had relapsed or progressed after previous treatment with selective estrogen receptor modulators (SERMs) and/or aromatase inhibitors (AIs). In some embodiments, the elacestrant is administered to the subject at a dose of from about 200 mg/day to about 500 mg/day. In some embodiments, the elacestrant is administered to the subject at a dose of about 200 mg/day, about 300 mg/day, about 400 mg/day, or about 500 mg/day. In some embodiments, the elacestrant is administered to the subject at a dose that is the maximum tolerated dose for the subject. In some embodiments, the method further comprises identifying the subject for treatment by measuring increased expression of one or more genes selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL. In some embodiments, the one or more genes is selected from AKT1, AKT2, BRAF, CDK4, CDK6, PIK3CA, PIK3R1, and MTOR. In some embodiments, the ratio of the concentration of elacestrant or a salt or solvate thereof in the tumor to the concentration of elacestrant or a salt or solvate thereof in plasma (T/P) following administration is at least about 15.

In another aspect, the disclosure relates to a method of treating a CDK4/6 inhibitor resistant estrogen receptor alpha-positive cancer in a subject having a wild-type estrogen receptor alpha and/or a mutant estrogen receptor alpha, the method comprising administering to the subject a therapeutically effective amount of elacestrant, or a pharmaceutically acceptable salt or solvate thereof, wherein the mutant estrogen receptor alpha comprises one or more mutations selected from the group consisting of D538G, Y537X₁, L536X₂, P535H, V534E, S463P, V392I, E380Q and combinations thereof, wherein: X₁ is S, N, or C; and X₂ is R or Q.

Embodiments of this aspect of the invention may include one or more of the following optional features. In some embodiments, the CDK4/6 inhibitor resistant estrogen receptor alpha positive cancer is resistant to palbociclib, ribociclib, abemaciclib, or a combination thereof. In some embodiments, the CDK4/6 inhibitor resistant estrogen receptor alpha positive cancer is resistant to palbociclib. In some embodiments, the CDK4/6 inhibitor resistant estrogen receptor alpha positive cancer is resistant to ribociclib. In some embodiments, the CDK4/6 inhibitor resistant estrogen receptor alpha positive cancer is resistant to abemaciclib. In some embodiments, the cancer is resistant to a drug selected from the group consisting of anti-estrogens, aromatase inhibitors, and combinations thereof. In some embodiments, the anti-estrogens are selected from the group consisting of tamoxifen, toremifene and fulvestrant and the aromatase inhibitors are selected from the group consisting of exemestane, letrozole and anastrozole. In some embodiments, the CDK4/6 inhibitor resistant estrogen receptor alpha-positive cancer is selected from the group consisting of breast cancer, uterine cancer, ovarian cancer, and pituitary cancer. In some embodiments, the cancer is advanced or metastatic breast cancer. In some embodiments, the cancer is breast cancer. In some embodiments, the subject is a post-menopausal woman. In some embodiments, the subject is a pre-menopausal woman. In some embodiments, the subject is a post-menopausal woman who had relapsed or progressed after previous treatment with SERMs and/or AIs. In some embodiments, the subject expresses at least one mutant estrogen receptor alpha selected from the group consisting of D538G, Y537S, Y537N, Y537C, E380Q, S463P, L536R, L536Q, P535H, V392I and V534E. In some embodiments, the mutation includes Y537S. In some embodiments, the mutation includes D538G. In some embodiments, the method further comprises identifying the subject for treatment by measuring increased expression of one or more genes selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL. In some embodiments, the one or more genes is selected from AKT1, AKT2, BRAF, CDK4, CDK6, PIK3CA, PIK3R1, and MTOR. In some embodiments, the elacestrant is administered to the subject at a dose of from about 200 to about 500 mg/day. In some embodiments, the elacestrant is administered to the subject at a dose of about 200 mg, about 300 mg, about 400 mg, or about 500 mg. In some embodiments, the elacestrant is administered to the subject at a dose of about 300 mg/day. In some embodiments, the ratio of the concentration of elacestrant or a salt or solvate thereof in the tumor to the concentration of elacestrant or a salt or solvate thereof in plasma (T/P) following administration is at least about 15.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF FIGURES

The following figures are provided by way of example and are not intended to limit the scope of the claimed invention.

FIG. 1A. The generation of resistance to palbociclib in the ESR1:wild-type LTED cell lines is plotted for the palbociclib sensitive (palbo^{S}) and palbociclib resistant (palbo^{R}) cell lines.

FIG. 1B. The percent growth inhibition (normalized to control as 100%) with respect to the Log[Palbociclib(μM)] is plotted for the palbo^{S} and palbo^{R} ESR1:wild-type cell lines.

FIG. 1C. Colony formation assay pictures of the palbo^{S} and palbo^{R} ESR1:wild-type cell lines are provided for the control and after treatment with palbociclib (500 nM).

FIG. 1D. A Western blot illustrating the LTED, LTED+palbo, LTED− palbo^{R}, and LTED− palbo^{R}+palbo models having an ESR1:wild-type gene.

FIG. 2A. The generation of resistance to palbociclib in the ESR1:D538G LTED cell lines plotted for the palbociclib sensitive (palbo^{S}) and palbociclib resistant (palbo^{R}) cell lines.

FIG. 2B. The percent growth inhibition (normalized to control as 100%) with respect to the Log[Palbociclib(μM)] are plotted for the palbo^{S} and palbo^{R} ESR1:D538G cell lines.

FIG. 2C. Colony formation assay pictures of the palbo^{S} and palbo^{R} ESR1:D538G cell lines are provided for the control and after treatment with palbociclib (500 nM).

FIG. 2D. A Western blot illustrating the LTED, LTED+palbo, LTED− palbo^{R}, and LTED− palbo^{R}+palbo models having an ESR1:D538G mutation.

FIG. 3A. The generation of resistance to palbociclib in the ESR1:Y537S LTED cell lines is plotted for the palbociclib sensitive (palbo^{S}) and palbociclib resistant (palbo^{R}) cell lines.

FIG. 3B. The percent growth inhibition (normalized to control as 100%) with respect to the Log[Palbociclib(μM)] is plotted for the palbo^{S} and palbo^{R} ESR1:Y537S cell lines.

FIG. 3C. Colony formation assay pictures of the palbo^{S} and palbo^{R} ESR1:Y537S cell lines are provided for the control and after treatment with palbociclib (500 nM).

FIG. 3D. A Western blot illustrating the LTED, LTED+palbo, LTED− palbo^{R}, and LTED− palbo^{R}+palbo models having an ESR1:Y537S mutation.

FIG. 4A. The generation of resistance to ribociclib in the ESR1:wild-type LTED cell lines is plotted for the ribociclib sensitive (ribo^{S}) and ribociclib resistant (ribo^{R}) cell lines.

FIG. 4B. The generation of resistance to abemaciclib in the ESR1:wild-type LTED cell lines is plotted for the abemaciclib sensitive (abema^{S}) and abemaciclib resistant (abema^{R}) cell lines.

FIG. 4C. The percent growth inhibition (normalized to control as 100%) with respect to the Log[Ribociclib(μM)] is plotted for the ribo^{S} and ribo^{R} ESR1:wild-type cell lines.

FIG. 4D. Colony formation assay pictures of the ribo^{S} and ribo^{R} ESR1:wild-type cell lines are provided for the control and after treatment with ribociclib (500 nM).

FIG. 4E. The percent growth inhibition (normalized to control as 100%) with respect to the Log[Abemaciclib(μM)] is plotted for the abema^{S} and abema^{R} ESR1:wild-type cell lines.

FIG. 4F. Colony formation assay pictures of the abema^{S} and abema^{R} ESR1:wild-type cell lines provided for the control and after treatment with abemaciclib (500 nM).

FIG. 5A. The generation of resistance to ribociclib in the ESR1:D538G LTED cell lines is plotted for the ribociclib sensitive (ribo^{S}) and ribociclib resistant (ribo^{R}) cell lines.

FIG. 5B. The generation of resistance to abemaciclib in the ESR1:D538G LTED cell lines is plotted for the abemaciclib sensitive (abema^{S}) and abemaciclib resistant (abema^{R}) cell lines.

FIG. 5C. The percent growth inhibition (normalized to control as 100%) with respect to the Log[Ribociclib(μM)] is plotted for the ribo^{S} and ribo^{R} ESR1:D538G cell lines.

FIG. 5D. Colony formation assay pictures of the ribo^{S} and ribo^{R} ESR1:D538G cell lines are provided for the control and after treatment with ribociclib (500 nM).

FIG. 5E. The percent growth inhibition (normalized to control as 100%) with respect to the Log[Abemaciclib(μM)] plotted for the abema^{S} and abema^{R} ESR1:D538G cell lines.

FIG. 5F. Colony formation assay pictures of the abema^{S} and abema^{R} ESR1:D538G cell lines provided for the control and after treatment with abemaciclib (500 nM).

FIG. 6A. The generation of resistance to ribociclib in the ESR1:Y537S LTED cell lines is plotted for the ribociclib sensitive (ribo^{S}) and ribociclib resistant (ribo^{R}) cell lines.

FIG. 6B. The generation of resistance to abemaciclib in the ESR1:Y537S LTED cell lines is plotted for the abemaciclib sensitive (abema^{S}) and abemaciclib resistant (abema^{R}) cell lines.

FIG. 6C. The percent growth inhibition (normalized to control as 100%) with respect to the Log[Ribociclib(μM)] is plotted for the ribo^{S} and ribo^{R} ESR1:Y537S cell lines.

FIG. 6D. Colony formation assay pictures of the ribo^{S} and ribo^{R} ESR1:Y537S cell lines are provided for the control and after treatment with ribociclib (500 nM).

FIG. 6E. The percent growth inhibition (normalized to control as 100%) with respect to the Log[Abemaciclib(μM)] is plotted for the abema^{S} and abema^{R} ESR1:Y537S cell lines.

FIG. 6F. Colony formation assay pictures of the abema^{S} and abema^{R} ESR1:Y537S cell lines are provided for the control and after treatment with abemaciclib (500 nM).

FIG. 7A. The EC₅₀ (nM) values are provided and the percent growth inhibition is plotted with respect to the Log[Elacestrant (nM)] for the ESR1:wild-type CDK4/6 inhibitor sensitive, ESR1:wild-type palbociclib^{R}, ESR1:wild-type ribociclib^{R}, and ESR1:wild-type abemaciclib^{R} cell lines.

FIG. 7B. The EC₅₀ (nM) values are provided and the percent growth inhibition is plotted with respect to the Log[Elacestrant (nM)] for the ESR1:D538G CDK4/6 inhibitor sensitive, ESR1:D538G palbociclib^{R}, ESR1:D538G ribociclib^{R}, and ESR1:D538G abemaciclib^{R} cell lines.

FIG. 7C. The EC₅₀ (nM) values are provided and the percent growth inhibition is plotted with respect to the Log[Elacestrant (nM)] for the ESR1:Y537S CDK4/6 inhibitor sensitive, ESR1:Y537S palbociclib^{R}, ESR1:Y537S ribociclib^{R}, and ESR1:Y537S abemaciclib^{R} cell lines.

FIG. 8A. The colony formation assay pictures in the top row visualize the growth for the control ESR1:wild-type CDK4/6 inhibitor sensitive, ESR1:wild-type palbociclib^{R}, ESR1:wild-type ribociclib^{R}, and ESR1:wild-type abemaciclib^{R} cell lines while the pictures in the bottom row visualize the cell growth for the control ESR1:wild-type CDK4/6 inhibitor sensitive, ESR1:wild-type palbociclib^{R}, ESR1:wild-type ribociclib^{R}, and ESR1:wild-type abemaciclib^{R} cell lines after being treated with elacestrant (300 nM).

FIG. 8B. The colony formation assay pictures in the top row visualize the growth for the control ESR1:D538G CDK4/6 inhibitor sensitive, ESR1:D538G palbociclib^{R}, ESR1:D538G ribociclib^{R}, and ESR1:D538G abemaciclib^{R} cell lines while the pictures in the bottom row visualize the cell growth for the control ESR1:D538G CDK4/6 inhibitor sensitive, ESR1:D538G palbociclib^{R}, ESR1:D538G ribociclib^{R}, and ESR1:D538G abemaciclib^{R} cell lines after being treated with elacestrant (300 nM).

FIG. 8C. The colony formation assay pictures in the top row visualize the growth for the control ESR1:Y537S CDK4/6 inhibitor sensitive, ESR1:Y537S palbociclib^{R}, ESR1:Y537A ribociclib^{R}, and ESR1:Y537S abemaciclib^{R} cell lines while the pictures in the bottom row visualize the cell growth for the control ESR1:Y537S CDK4/6 inhibitor sensitive, ESR1:Y537S palbociclib^{R}, ESR1:Y537S ribociclib^{R}, and ESR1:Y537S abemaciclib^{R} cell lines after being treated with elacestrant (300 nM).

FIG. 9A. Average tumor volumes over time in athymic nude mice implanted with the WHIM43-HI PDX xenograft with an ESR1:D538G mutation were treated with a vehicle control, palbociclib, fulvestrant (3 mg/dose), and elacestrant (30 and 60 mg/kg).

FIG. 9B. The quantification of ERα protein level was determined by plotting ERα/vinculin (normalized to control) against treatment of the WHIM43-HI PDX xenograft model having an ESR1:D538G mutation with a vehicle control, palbociclib, fulvestrant (3 mg/dose), and elacestrant (30 and 60 mg/kg).

FIG. 9C. The quantification of E2F1 protein level was determined by plotting E2F1/vinculin (normalized to control) against treatment of the WHIM43-HI PDX xenograft model having an ESR1:D538G mutation with a vehicle control, palbociclib, fulvestrant (3 mg/dose), and elacestrant (30 and 60 mg/kg).

FIG. 9D. The quantification of CCNE1 protein level was determined by plotting CCNE1/vinculin (normalized to control) against treatment of the WHIM43-HI PDX xenograft model having an ESR1:D538G mutation with a vehicle control, palbociclib, fulvestrant (3 mg/dose), and elacestrant (30 and 60 mg/kg).

FIG. 9E. The PgR mRNA levels were determined by plotting fold change (normalized to control) against treatment of the WHIM43-HI PDX xenograft model having an ESR1:D538G mutation with a vehicle control, palbociclib, fulvestrant (3 mg/dose), and elacestrant (30 and 60 mg/kg).

FIG. 9F. A Western blot illustrating the WHIM43-HI PDX xenograft model having an ESR1:D538G mutation treated with a vehicle control, palbociclib, fulvestrant (3 mg/dose), and elacestrant (30 and 60 mg/kg) is provided.

FIG. 10A. The quantification of progesterone receptor (PgR) in tumor cell models having an ESR1:wild-type mutation was determined by plotting PgR mRNA levels (normalized to control) against treatment of the palbo^{S} and palbo^{R} cell lines.

FIG. 10B. The quantification of trefoil factor 1 (TFF1) in tumor cell models having an ESR1:wild-type mutation was determined by plotting TFF1 mRNA levels (normalized to control) against treatment of the palbo^{S} and palbo^{R} cell lines.

FIG. 10C. The quantification of growth regulated by estrogen (GREB1) in tumor cell models having an ESR1:wild-type mutation was determined by plotting GREB1 mRNA levels (normalized to control) against treatment of the palbo^{S} and palbo^{R} cell lines.

FIG. 11A. The quantification of progesterone receptor (PgR) in tumor cell models having an ESR1:D538G mutation was determined by plotting PgR mRNA levels (normalized to control) against treatment of the palbo^{S} and palbo^{R} cell lines.

FIG. 11B. The quantification of trefoil factor 1 (TFF1) in tumor cell models having an ESR1:D538G mutation was determined by plotting TFF1 mRNA levels (normalized to control) against treatment of the palbo^{S} and palbo^{R} cell lines.

FIG. 11C. The quantification of growth regulated by estrogen (GREB1) in tumor cell models having an ESR1:D538G mutation was determined by plotting GREB1 mRNA levels (normalized to control) against treatment of the palbo^{S} and palbo^{R} cell lines.

FIG. 12A. The quantification of progesterone receptor (PgR) in tumor cell models having an ESR1:Y537S mutation was determined by plotting PgR mRNA levels (normalized to control) against treatment of the palbo^{S} and palbo^{R} cell lines.

FIG. 12B. The quantification of trefoil factor 1 (TFF1) in tumor cell models having an ESR1:Y537S mutation was determined by plotting TFF1 mRNA levels (normalized to control) against treatment of the palbo^{S} and palbo^{R} cell lines.

FIG. 12C. The quantification of growth regulated by estrogen (GREB1) in tumor cell models having an ESR1:Y537S mutation was determined by plotting GREB1 mRNA levels (normalized to control) against treatment of the palbo^{S} and palbo^{R} cell lines.

FIG. 13. The tumor volume of the ST941-HI PDX xenograft model (treatment-naïve) is plotted against the days of treatment, where the model was treated with vehicle or a combination of fulvestrant and palbociclib (the fulvestrant 3 mg/dose data is from a separate study). The tumors from the fulvestrant and palbociclib arm were then re-implanted into another study (ST941-HI palbociclib-treated; first passage) and then subsequently treated with the vehicle, fulvestrant (3 mg/dose), palbociclib (25 mg/kg), and elacestrant (30 mg/kg) to demonstrate that elacestrant is still effective at inhibiting tumor growth in the PDX model previously treated with a combination of fulvestrant and palbociclib.

### DETAILED DESCRIPTION OF THE INVENTION

As used herein, elacestrant or “RAD1901” is an orally bioavailable selective estrogen receptor degrader (SERD) and has the following chemical structure:

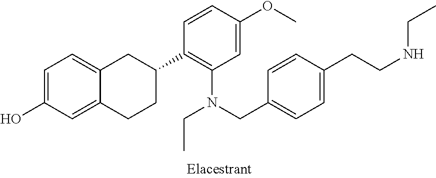 [SMILES: CCNCCc1ccc(CN(CC)c2cc(OC)ccc2[C@@H]2CCc3cc(O)ccc3C2)cc1]

including salts, solvates (e.g. hydrate), and prodrugs thereof. Preclinical data have demonstrated elacestrant is effective in inhibiting tumor growth in models of ER+ breast cancer with both wild-type and mutant ESR1. In some embodiments described herein, elacestrant is administered as the bis-hydrochloride (.2HCl) salt having the following chemical structure:

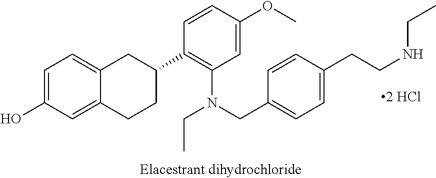 [SMILES: CCNCCc1ccc(CN(CC)c2cc(OC)ccc2[C@@H]2CCc3cc(O)ccc3C2)cc1]

In postmenopausal women, the current standard of care for ER+ cancers, such as breast cancer, involves inhibiting the ER pathway by: 1) inhibiting the synthesis of estrogen (aromatase inhibitors (AI)); 2) directly binding to ER and modulating its activity using SERMs (e.g., tamoxifen); and/or 3) directly binding to ER and causing receptor degradation using SERDs (e.g., fulvestrant). In premenopausal women, the current standard of care would additionally include ovarian suppression through oophorectomy or a luteinizing hormone-releasing hormone (LHRH) agonist. The addition of a cyclin-dependent kinase 4/6 (CDK4/6) inhibitor to an endocrine agent has been shown in some cases to roughly double the Progression Free Survival (PFS) where these types of results have led to the approval and use of certain CDK4/6 inhibitors in combination with either AIs in the first-line metastatic setting or in combination with fulvestrant in the second-line metastatic setting. Data showing the trends of ESR1s to mutate and become resistant to AIs and/or CDK4/6 inhibitors highlights the need for new and improved orally-bioavailable endocrine therapies that have efficacy against wild-type ESR1 and all ESR1 mutations.

The addition of a cyclin-dependent kinase 4/6 (CDK4/6) inhibitor to an endocrine agent roughly doubles Progression Free Survival (PFS), and this has led to the approval and use of certain CDK4/6 inhibitors in combination with either AI in the first-line metastatic setting or in combination with fulvestrant, in either the first- or second-line metastatic setting. In the methods described herein, elacestrant is shown to induce dose-dependent long-term growth inhibition in CDK4/6 inhibitor-resistant cancer cell lines regardless of prior treatment history or ESR1 mutant status. Elacestrant (30 mg/kg) also demonstrated tumor growth inhibition in vivo in PDX models that were previously treated with palbociclib (>100 days) and/or were de novo resistant to palbociclib. In addition, elacestrant demonstrated growth inhibitory activity in vitro and in vivo in several CDK4/6 inhibitor-resistant models that demonstrated several molecular markers of CDK4/6 inhibitor resistance such as, but not limited to, loss of Rb, overexpression of Cyclin E1, overexpression of E2F1, overexpression of Cyclin D1, and overexpression of CDK6.

Elacestrant (30 mg/kg) was demonstrated to degrade ER, reduce E2F1 expression, and reduce Cyclin E1 expression in the WHIM43-HI PDX model (palbociclib-resistant/Rb null). The characterization of CDK4/6 inhibitor resistance demonstrated that in these resistant cell lines ER, ER signaling, and importantly, ER-driven proliferation was retained. Therefore, the studies herein indicating the effective use of elacestrant as a treatment for CDK4/6 inhibitor-resistant cancers having various types of ESR1 mutations is a promising discovery.

### Definitions

As used herein, the following definitions shall apply unless otherwise indicated.

As used herein, the terms “RAD1901” and “elacestrant” refer to the same chemical compound and are used interchangeably.

“Inhibiting growth” of an ERα-positive tumor as used herein may refer to slowing the rate of tumor growth, or halting tumor growth entirely.

“Tumor regression” or “regression” of an ERα-positive tumor as used herein may refer to reducing the maximum size of a tumor. In certain embodiments, administration of a combination as described herein, or solvates (e.g., hydrate) or salts thereof may result in a decrease in tumor size versus baseline (i.e., size prior to initiation of treatment), or even eradication or partial eradication of a tumor. Accordingly, in certain embodiments the methods of tumor regression provided herein may be alternatively characterized as methods of reducing tumor size versus baseline.

“Tumor” as used herein is a malignant tumor, and is used interchangeably with “cancer.”

“Estrogen receptor alpha” or “ERα” as used herein refers to a polypeptide comprising, consisting of, or consisting essentially of the wild-type ERα amino acid sequence, which is encoded by the gene ESR1.

A tumor that is “positive for estrogen receptor alpha,” “ERα-positive,” “ER+,” or “ERα+” as used herein refers to a tumor in which one or more cells express at least one isoform of ERα.

### Methods of Treatment

In some embodiments, the disclosure relates to a method of inhibiting and degrading a CDK4/6 inhibitor resistant estrogen receptor alpha positive cancer in a subject comprising administering to the subject a therapeutically effective amount of elacestrant, or a pharmaceutically acceptable salt or solvate thereof.

In other embodiments, the disclosure relates to a method of treating a CDK4/6 inhibitor resistant estrogen receptor alpha-positive cancer in a subject having a wild-type estrogen receptor alpha and/or a mutant estrogen receptor alpha, the method comprising administering to the subject a therapeutically effective amount of elacestrant, or a pharmaceutically acceptable salt or solvate thereof, wherein the mutant estrogen receptor alpha comprises one or more mutations selected from the group consisting of D538G, Y537X₁, L536X₂, P535H, V534E, S463P, V392I, E380Q and combinations thereof, wherein: X₁ is S, N, or C; and X₂ is R or Q.

### Administration of Elacestrant

Elacestrant or solvates (e.g., hydrate) or salts thereof, when administered to a subject, have a therapeutic effect on one or more cancers or tumors. Tumor growth inhibition or regression may be localized to a single tumor or to a set of tumors within a specific tissue or organ, or may be systemic (i.e., affecting tumors in all tissues or organs).

As elacestrant is known to preferentially bind ERα versus estrogen receptor beta (ERβ), unless specified otherwise, estrogen receptor, estrogen receptor alpha, ERα, ER, and wild-type ERα are used interchangeably herein. In certain embodiments, ER+ cells overexpress ERα. In certain embodiments, the patient has one or more cells within the tumor expressing one or more forms of ERβ. In certain embodiments, the ERα-positive tumor and/or cancer is associated with breast, uterine, ovarian, or pituitary cancer. In certain of these embodiments, the patient has a tumor located in breast, uterine, ovarian, or pituitary tissue. In those embodiments where the patient has a tumor located in the breast, the tumor may be associated with luminal breast cancer that may or may not be positive for HER2, and for HER2+ tumors, the tumors may express high or low HER2. In other embodiments, the patient has a tumor located in another tissue or organ (e.g., bone, muscle, brain), but is nonetheless associated with breast, uterine, ovarian, or pituitary cancer (e.g., tumors derived from migration or metastasis of breast, uterine, ovarian, or pituitary cancer). Accordingly, in certain embodiments of the tumor growth inhibition or tumor regression methods provided herein, the tumor being targeted is a metastatic tumor and/or the tumor has an overexpression of ER in other organs (e.g., bones and/or muscles). In certain embodiments, the tumor being targeted is a brain tumor and/or cancer. In certain embodiments, the tumor being targeted may be more sensitive to a treatment of elacestrant than treatment with another SERD (e.g., fulvestrant, TAS-108 (SR16234), ZK191703, RU58668, GDC-0810 (ARN-810), GW5638/DPC974, SRN-927, and AZD9496), Her2 inhibitors (e.g., trastuzumab, lapatinib, ado-trastuzumab emtansine, and/or pertuzumab), chemo therapy (e.g., abraxane, adriamycin, carboplatin, cytoxan, daunorubicin, doxil, ellence, fluorouracil, gemzar, helaven, lxempra, methotrexate, mitomycin, micoxantrone, navelbine, taxol, taxotere, thiotepa, vincristine, and xeloda), aromatase inhibitor (e.g., anastrozole, exemestane, and letrozole), selective estrogen receptor modulators (e.g., tamoxifen, raloxifene, lasofoxifene, and/or toremifene), angiogenesis inhibitor (e.g., bevacizumab), and/or rituximab.

In addition to demonstrating the ability of elacestrant to inhibit tumor growth in tumors expressing wild-type ERα, elacestrant exhibits the ability to inhibit the growth of tumors expressing a mutant form of ERα, namely Y537S ERα. Computer modeling evaluations of examples of ERα mutations showed that none of these mutations were expected to impact the LBD or specifically hinder elacestrant binding, e.g., ERα having one or more mutants selected from the group consisting of ERα with Y537X mutant wherein X is S, N, or C, ERα with D538G mutant, and ERα with S463P mutant. Based on these results, methods are provided herein for inhibiting growth or producing regression of a tumor that is positive for ERα having one or more mutants within the ligand-binding domain (LBD), selected from the group consisting of Y537X1 wherein X1 is S, N, or C, D538G, L536X2 wherein X2 is R or Q, P535H, V534E, S463P, V392I, E380Q, especially Y537S ERα, in a subject with cancer by administering to the subject a therapeutically effective amount of elacestrant or solvates (e.g., hydrate) or salts thereof “Mutant ERα” as used herein refers to ERα comprising one or more substitutions or deletions, and variants thereof comprising, consisting of, or consisting essentially of an amino acid sequence with at least 80%, at least 85%, at least 90%, at least 95%, at least 97%, at least 98%, at least 99%, or at least 99.5% identity to the amino acid sequence of ERα.

In addition to inhibiting breast cancer tumor growth in an animal xenograft model, elacestrant exhibits significant accumulation within tumor cells, and is capable of penetrating the blood-brain barrier. The ability to penetrate the blood-brain barrier was confirmed by showing that elacestrant administration significantly prolonged survival in a brain metastasis xenograft model. Accordingly, in certain embodiments of the tumor growth inhibition or tumor regression methods provided herein, the ERα-positive tumor being targeted is located in the brain or elsewhere in the central nervous system. In certain of these embodiments, the ERα-positive tumor is primarily associated with brain cancer. In other embodiments, the ERα-positive tumor is a metastatic tumor that is primarily associated with another type of cancer, such as breast, uterine, ovarian, or pituitary cancer, or a tumor that has migrated from another tissue or organ. In certain of these embodiments, the tumor is a brain metastases, such as breast cancer brain metastases (BCBM). In certain embodiments of the methods disclosed herein, elacestrant or solvates (e.g., hydrate) or salts thereof accumulate in one or more cells within a target tumor.

In certain embodiments of the methods disclosed herein, elacestrant or solvates (e.g., hydrate) or salts thereof preferably accumulate in tumor at a T/P (elacestrant concentration in tumor/elacestrant concentration in plasma) ratio of about 15 or higher, about 18 or higher, about 19 or higher, about 20 or higher, about 25 or higher, about 28 or higher, about 30 or higher, about 33 or higher, about 35 or higher, or about 40 or higher.

### Dosage

A therapeutically effective amount of a combination of elacestrant or solvates (e.g., hydrate) or salts thereof for use in the methods disclosed herein is an amount that, when administered over a particular time interval, results in achievement of one or more therapeutic benchmarks (e.g., slowing or halting of tumor growth, resulting in tumor regression, cessation of symptoms, etc.). The combination for use in the presently disclosed methods may be administered to a subject one time or multiple times. In those embodiments wherein the compounds are administered multiple times, they may be administered at a set interval, e.g., daily, every other day, weekly, or monthly. Alternatively, they can be administered at an irregular interval, for example on an as-needed basis based on symptoms, patient health, and the like. A therapeutically effective amount of the combination may be administered q.d. for one day, at least 2 days, at least 3 days, at least 4 days, at least 5 days, at least 6 days, at least 7 days, at least 10 days, or at least 15 days. Optionally, the status of the cancer or the regression of the tumor is monitored during or after the treatment, for example, by a FES-PET scan of the subject. The dosage of the combination administered to the subject can be increased or decreased depending on the status of the cancer or the regression of the tumor detected.

Ideally, the therapeutically effective amount does not exceed the maximum tolerated dosage at which 50% or more of treated subjects experience nausea or other toxicity reactions that prevent further drug administrations. A therapeutically effective amount may vary for a subject depending on a variety of factors, including variety and extent of the symptoms, sex, age, body weight, or general health of the subject, administration mode and salt or solvate type, variation in susceptibility to the drug, the specific type of the disease, and the like.

Examples of therapeutically effective amounts of a elacestrant or solvates (e.g., hydrate) or salts thereof for use in the methods disclosed herein include, without limitation, about 150 to about 1,500 mg, about 200 to about 1,500 mg, about 250 to about 1,500 mg, or about 300 to about 1,500 mg dosage q.d. for subjects having resistant ER-driven tumors or cancers; about 150 to about 1,500 mg, about 200 to about 1,000 mg or about 250 to about 1,000 mg or about 300 to about 1,000 mg dosage q.d. for subjects having both wild-type ER driven tumors and/or cancers and resistant tumors and/or cancers; and about 300 to about 500 mg, about 300 to about 550 mg, about 300 to about 600 mg, about 250 to about 500 mg, about 250 to about 550 mg, about 250 to about 600 mg, about 200 to about 500 mg, about 200 to about 550 mg, about 200 to about 600 mg, about 150 to about 500 mg, about 150 to about 550 mg, or about 150 to about 600 mg q.d. dosage for subjects having majorly wild-type ER driven tumors and/or cancers. In certain embodiments, the dosage of a compound of Formula I (e.g., elacestrant) or a salt or solvate thereof for use in the presently disclosed methods general for an adult subject may be approximately 200 mg, 400 mg, 30 mg to 2,000 mg, 100 mg to 1,500 mg, or 150 mg to 1,500 mg p.o., q.d. This daily dosage may be achieved via a single administration or multiple administrations.

Dosing of elacestrant in the treatment of breast cancer including resistant strains as well as instances expressing mutant receptor(s) are in the range of 100 mg to 1,000 mg per day. For example, elacestrant may be dosed at 100, 200, 300, 400, 500, 600, 700, 800, 900 or 1,000 mg per day. In particular, 200 mg, 400 mg, 500 mg, 600 mg, 800 mg and 1,000 mg per day are noted. The surprisingly long half-life of elacestrant in humans after PO dosing make this option particularly viable. Accordingly, the drug may be administered as 200 mg bid (400 mg total daily), 250 mg bid (500 mg total daily), 300 mg bid (600 mg total daily), 400 mg bid (800 mg daily) or 500 mg bid (1,000 mg total daily). In some embodiments, the dosing is oral.

In certain embodiments of the methods disclosed herein, elacestrant or a solvate (e.g., hydrate) or salt thereof preferably accumulate in tumor at a T/P (elacestrant concentration in tumor/elacestrant concentration in plasma) ratio of about 15 or higher, about 18 or higher, about 19 or higher, about 20 or higher, about 25 or higher, about 28 or higher, about 30 or higher, about 33 or higher, about 35 or higher, or about 40 or higher.

The elacestrant or solvates (e.g., hydrate) or salts thereof may be administered to a subject one time or multiple times. In those embodiments wherein the compounds are administered multiple times, they may be administered at a set interval, e.g., daily, every other day, weekly, or monthly. Alternatively, they can be administered at an irregular interval, for example on an as-needed basis based on symptoms, patient health, and the like.

### Formulation

In some embodiments, elacestrant or solvates (e.g., hydrate) or salts thereof are administered as part of a single formulation. For example, elacestrant or solvates (e.g., hydrate) or salts thereof are formulated in a single pill for oral administration or in a single dose for injection. In certain embodiments, administration of the compounds in a single formulation improves patient compliance.

In some embodiments, a formulation comprising elacestrant or solvates (e.g., hydrate) or salts thereof may further comprise one or more pharmaceutical excipients, carriers, adjuvants, and/or preservatives.

The elacestrant or solvates (e.g., hydrate) or salts thereof for use in the presently disclosed methods can be formulated into unit dosage forms, meaning physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times q.d.). When multiple daily doses are used, the unit dosage form can be the same or different for each dose. In certain embodiments, the compounds may be formulated for controlled release.

The elacestrant or solvates (e.g., hydrate) or salts thereof and salts or solvates for use in the presently disclosed methods can be formulated according to any available conventional method. Examples of preferred dosage forms include a tablet, a powder, a subtle granule, a granule, a coated tablet, a capsule, a syrup, a troche, an inhalant, a suppository, an injectable, an ointment, an ophthalmic ointment, an eye drop, a nasal drop, an ear drop, a cataplasm, a lotion and the like. In the formulation, generally used additives such as a diluent, a binder, an disintegrant, a lubricant, a colorant, a flavoring agent, and if necessary, a stabilizer, an emulsifier, an absorption enhancer, a surfactant, a pH adjuster, an antiseptic, an antioxidant and the like can be used. In addition, the formulation is also carried out by combining compositions that are generally used as a raw material for pharmaceutical formulation, according to the conventional methods. Examples of these compositions include, for example, (1) an oil such as a soybean oil, a beef tallow and synthetic glyceride; (2) hydrocarbon such as liquid paraffin, squalane and solid paraffin; (3) ester oil such as octyldodecyl myristic acid and isopropyl myristic acid; (4) higher alcohol such as cetostearyl alcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, a solid polyoxyethylene castor oil and polyoxyethylene polyoxypropylene block co-polymer; (8) water soluble macromolecule such as hydroxyethyl cellulose, polyacrylic acid, carboxyvinyl polymer, polyethyleneglycol, polyvinylpyrrolidone and methylcellulose; (9) lower alcohol such as ethanol and isopropanol; (10) multivalent alcohol such as glycerin, propyleneglycol, dipropyleneglycol and sorbitol; (11) a sugar such as glucose and cane sugar; (12) an inorganic powder such as anhydrous silicic acid, aluminum magnesium silicicate and aluminum silicate; and (13) purified water, and the like. Additives for use in the above formulations may include, for example, 1) lactose, corn starch, sucrose, glucose, mannitol, sorbitol, crystalline cellulose and silicon dioxide as the diluent; 2) polyvinyl alcohol, polyvinyl ether, methyl cellulose, ethyl cellulose, gum arabic, tragacanth, gelatine, shellac, hydroxypropyl cellulose, hydroxypropylmethyl cellulose, polyvinylpyrrolidone, polypropylene glycol-poly oxyethylene-block co-polymer, meglumine, calcium citrate, dextrin, pectin and the like as the binder; 3) starch, agar, gelatine powder, crystalline cellulose, calcium carbonate, sodium bicarbonate, calcium citrate, dextrin, pectic, carboxymethylcellulose/calcium and the like as the disintegrant; 4) magnesium stearate, talc, polyethyleneglycol, silica, condensed plant oil and the like as the lubricant; 5) any colorants whose addition is pharmaceutically acceptable is adequate as the colorant; 6) cocoa powder, menthol, aromatizer, peppermint oil, cinnamon powder as the flavoring agent; and 7) antioxidants whose addition is pharmaceutically accepted such as ascorbic acid or alpha-tophenol.

Elacestrant or solvates (e.g., hydrate) or salts thereof for use in the presently disclosed methods can be formulated into a pharmaceutical composition as any one or more of the active compounds described herein and a physiologically acceptable carrier (also referred to as a pharmaceutically acceptable carrier or solution or diluent). Such carriers and solutions include pharmaceutically acceptable salts and solvates of compounds used in the methods of the instant invention, and mixtures comprising two or more of such compounds, pharmaceutically acceptable salts of the compounds and pharmaceutically acceptable solvates of the compounds. Such compositions are prepared in accordance with acceptable pharmaceutical procedures such as described in Remington's Pharmaceutical Sciences, 17th edition, ed. Alfonso R. Gennaro, Mack Publishing Company, Eaton, Pa. (1985), which is incorporated herein by reference.

The term “pharmaceutically acceptable carrier” refers to a carrier that does not cause an allergic reaction or other untoward effect in patients to whom it is administered and are compatible with the other ingredients in the formulation. Pharmaceutically acceptable carriers include, for example, pharmaceutical diluents, excipients or carriers suitably selected with respect to the intended form of administration, and consistent with conventional pharmaceutical practices. For example, solid carriers/diluents include, but are not limited to, a gum, a starch (e.g., corn starch, pregelatinized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof. Pharmaceutically acceptable carriers may further comprise minor amounts of auxiliary substances such as wetting or emulsifying agents, preservatives or buffers, which enhance the shelf life or effectiveness of the therapeutic agent.

Elacestrant or solvates (e.g., hydrate) or salts thereof in a free form can be converted into a salt by conventional methods. The term “salt” used herein is not limited as long as the salt is formed with elacestrant or solvates (e.g., hydrate) or salts thereof and is pharmacologically acceptable; preferred examples of salts include a hydrohalide salt (for instance, hydrochloride, hydrobromide, hydroiodide and the like), an inorganic acid salt (for instance, sulfate, nitrate, perchlorate, phosphate, carbonate, bicarbonate and the like), an organic carboxylate salt (for instance, acetate salt, maleate salt, tartrate salt, fumarate salt, citrate salt and the like), an organic sulfonate salt (for instance, methanesulfonate salt, ethanesulfonate salt, benzenesulfonate salt, toluenesulfonate salt, camphorsulfonate salt and the like), an amino acid salt (for instance, aspartate salt, glutamate salt and the like), a quaternary ammonium salt, an alkaline metal salt (for instance, sodium salt, potassium salt and the like), an alkaline earth metal salt (magnesium salt, calcium salt and the like) and the like. In addition, hydrochloride salt, sulfate salt, methanesulfonate salt, acetate salt and the like are preferred as “pharmacologically acceptable salt” of the compounds according to the present invention.

Isomers of elacestrant or solvates (e.g., hydrate) or salts thereof (e.g., geometric isomers, optical isomers, rotamers, tautomers, and the like) can be purified using general separation means, including for example recrystallization, optical resolution such as diastereomeric salt method, enzyme fractionation method, various chromatographies (for instance, thin layer chromatography, column chromatography, glass chromatography and the like) into a single isomer. The term “a single isomer” herein includes not only an isomer having a purity of 100%, but also an isomer containing an isomer other than the target, which exists even through the conventional purification operation. A crystal polymorph sometimes exists for elacestrant or solvates (e.g., hydrate) or salts thereof and/or fulvestrant, and all crystal polymorphs thereof are included in the present invention. The crystal polymorph is sometimes single and sometimes a mixture, and both are included herein.

In certain embodiments, elacestrant or solvates (e.g., hydrate) or salts thereof may be in a prodrug form, meaning that it must undergo some alteration (e.g., oxidation or hydrolysis) to achieve its active form. Alternative, elacestrant or solvates (e.g., hydrate) or salts thereof may be a compound generated by alteration of a parental prodrug to its active form.

### Administration Route

Administration routes of elacestrant or solvates (e.g., hydrate) or salts thereof include but not limited to topical administration, oral administration, intradermal administration, intramuscular administration, intraperitoneal administration, intravenous administration, intravesical infusion, subcutaneous administration, transdermal administration, and transmucosal administration. In some embodiments, the administration route is oral.

### Gene Profiling

In certain embodiments, the methods of tumor growth inhibition or tumor regression provided herein further comprise gene profiling the subject, wherein the gene to be profiled is one or more genes selected from the group consisting of ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL. In other embodiments, the gene to be profiled is one or more genes selected from the group consisting of AKT1, AKT2, BRAF, CDK4, CDK6, PIK3CA, PIK3R1, and MTOR.

In some embodiments, this invention provides a method of treating a subpopulation of breast cancer patients wherein said sub-population has increased expression of one or more of the genes disclosed supra, and treating said sub-population with an effective dose of elacestrant or solvates (e.g., hydrate) or salts thereof according to the dosing embodiments as described in this disclosure.

### Dose Adjusting

In addition to establishing the ability of elacestrant to inhibit tumor growth, elacestrant inhibits estradiol binding to ER in the uterus and pituitary. In these experiments, estradiol binding to ER in uterine and pituitary tissue was evaluated by FES-PET imaging. After treatment with elacestrant, the observed level of ER binding was at or below background levels. These results establish that the antagonistic effect of elacestrant on ER activity can be evaluated using real-time scanning. Based on these results, methods are provided herein for monitoring the efficacy of treatment elacestrant or solvates (e.g., hydrate) or salts thereof in a combination therapy disclosed herein by measuring estradiol-ER binding in one or more target tissues, wherein a decrease or disappearance in binding indicates efficacy.

Further provided are methods of adjusting the dosage of elacestrant or solvates (e.g., hydrate) or salts thereof in a combination therapy disclosed herein based on estradiol-ER binding. In certain embodiments of these methods, binding is measured at some point following one or more administrations of a first dosage of the compound. If estradiol-ER binding is not affected or exhibits a decrease below a predetermined threshold (e.g., a decrease in binding versus baseline of less than 5%, less than 10%, less than 20%, less than 30%, or less than 50%), the first dosage is deemed to be too low. In certain embodiments, these methods comprise an additional step of administering an increased second dosage of the compound. These steps can be repeated, with dosage repeatedly increased until the desired reduction in estradiol-ER binding is achieved. In certain embodiments, these steps can be incorporated into the methods of inhibiting tumor growth provided herein. In these methods, estradiol-ER binding can serve as a proxy for tumor growth inhibition, or a supplemental means of evaluating growth inhibition. In other embodiments, these methods can be used in conjunction with the administration of elacestrant or solvates (e.g., hydrate) or salts thereof for purposes other than inhibition of tumor growth, including for example inhibition of cancer cell proliferation.

In certain embodiments, the methods provided herein for adjusting the dosage of elacestrant or salt or solvate (e.g., hydrate) thereof in a combination therapy comprise:

(1) administering a first dosage of elacestrant or salt or solvate (e.g., hydrate) thereof (e.g., about 350 to about 500 or about 200 to about 600 mg/day) for 3, 4, 5, 6, or 7 days;

(2) detecting estradiol-ER binding activity; wherein:
(i) if the ER binding activity is not detectable or is below a predetermined threshold level, continuing to administer the first dosage (i.e., maintain the dosage level); or
(ii) if the ER binding activity is detectable or is above a predetermined threshold level, administering a second dosage that is greater than the first dosage (e.g., the first dosage plus about 50 to about 200 mg) for 3, 4, 5, 6, or 7 days, then proceeding to step (3);

(3) detecting estradiol-ER binding activity; wherein
(i) if the ER binding activity is not detectable or is below a predetermined threshold level, continuing to administer the second dosage (i.e., maintain the dosage level); or
(ii) if the ER binding activity is detectable or is above a predetermined threshold level, administering a third dosage that is greater than the second dosage (e.g., the second dosage plus about 50 to about 200 mg) for 3, 4, 5, 6, or 7 days, then proceeding to step (4);

(4) repeating the steps above through a fourth dosage, fifth dosage, etc., until no ER binding activity is detected.

In certain embodiments, the invention includes the use of PET imaging to detect and/or dose ER sensitive or ER resistant cancers.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### Examples

### Materials and Methods

### Test Compounds

Elacestrant used in the examples below was (6R)-6-(2-(N-(4-(2-(ethylamino)ethyl)benzyl)-N-ethylamino)-4-methoxyphenyl)-5,6,7,8-tetrahydronaphthalen-2-ol dihydrochloride, manufactured by, for example, IRIX Pharmaceuticals, Inc. (Florence, S.C.). Elacestrant was stored as a dry powder, formulated for use as a homogenous suspension in 0.5% (w/v) methylcellulose in deionized water, and for animal models was administered p.o. Tamoxifen, raloxifene and estradiol (E2) were obtained from Sigma-Aldrich (St. Louis, Mo.), and administered by subcutaneous injection. Fulvestrant was obtained from Tocris Biosciences (Minneapolis, Minn.) and administered by subcutaneous injection. Other laboratory reagents were purchased from Sigma-Aldrich unless otherwise noted.

### Generation of CDK4/6 Inhibitor Resistance

### In Vitro

HCC1428-LTED-CDK4/6^{R} cells, MCF7-Y537S-CDK4/6^{R}, and MCF7-D538G-CDK4/6^{R} were generated by exposing HCC1428-LTED cells to increasing concentrations of the appropriate CDK4/6i with current maintenance of these cells at 500 nM of palbociclib and ribociclib for Palbo^{R} and Ribo^{R} cells respectively, and 250 nM of abemaciclib for Abema^{R} cells.

### In Vivo

ST941-HI patient-derived xenograft fragments were implanted into athymic-nude mice. Tumors were measured twice/wk with Vernier calipers; volumes were calculated using the formula: (L*W2)*0.5. The tumors were treated with Vehicle, Fulvestrant (3 mg/dose/week)+palbociclib (25 mg/kg daily), and RAD1901 (30 mg/kg daily). Tumors growing in the presence of fulvestrant (3 mg/dose/week)+palbociclib (25 mg/kg daily) were allowed to grow>1500 mm³ and then harvested and retransplanted into a new cohort of mice considered passage (P1).

### PDX Models

Tumors were passaged as fragments into athymic nude mice (Nu (NCR)-Foxn1nu). WHIM43 patient-derived xenograft fragments were implanted into ovariectomized mice without estradiol supplementation (Horizon). All mice were housed in pathogen-free housing in individually ventilated cages with sterilized and dust-free bedding cobs, access to sterilized food and water ad libitum, under a light dark cycle (12-14 hour circadian cycle of artificial light) and controlled room temperature and humidity. Tumors were measured twice/wk with Vernier calipers; volumes were calculated using the formula: (L*W2)*0.52. Elacestrant and palbociclib was administered orally, daily for the duration of study. Fulvestrant was administered once/week subcutaneously.

### Quantitative Real-Time PCR (RT-qPCR)

### In Vivo Xenograft Models

End of study tumors were pulverized with the cryoPREP™ Impactor (Covaris) and total RNA was extracted with the RNeasy mini kit (Qiagen). qPCR was performed using the Taqman Fast Virus 1-Step Master Mix and TaqMan™ probes (Applied Biosystems) The Ct values were analyzed to assess relative changes in expression of PgR (progesterone receptor) mRNA, with GAPDH as an internal control, using the 2-ΔΔCT method.

### In Vitro Xenograft Models

At the end of treatment, cells were lysed with the lysis buffer from the 1-step Cells-to-Ct kit and the lysates were processed according to the manufacturer's instructions. qPCR was performed using the 1-step master mix and TaqMan™ probes (Applied Biosystems). The Ct values were analyzed to assess relative changes in expression of PgR (progesterone receptor) mRNA, tff1 (trefoil factor 1) mRNA, and GREB1 (growth regulated by estrogen) mRNA with GAPDH as an internal control, using the 2-ΔΔCT method.

### Proliferation Assays

Cells were plated at a density of 5000 cells/well and treated the next day with respective treatments. Viability was measured after 7 days of incubation with drug and data was normalized to the control values as 100%. Data is graphed as % growth inhibition relative to control at day 7.

### Colony Formation Assay

Cells were plated at a density of 1000-10000 cells/well and allowed to grow for 2-5 wks depending on the cell line. Treatments were performed in triplicate; media and compound was replaced weekly. At the end of treatment, cells were fixed in paraformaldehyde and stained with crystal violet for visualization.

### Western Blot Analysis

Cells or tumors were harvested post-dosing) and protein expression analyzed using standard practice and antibodies as follows: ERa, PR, E2F1, CCNE1, CCNE2, CCND1, Rb, pRb, CDK2, CDK4, CDK6 (Cell Signaling Technologies, Cat #13258; #3153; #3742, #4129, #4132, #2978, #9309, #8516, #2546, #12790, #13331), p107, p130 (Abcam: ab168458, ab6545) and Vinculin: Sigma-Aldrich, #v9131. Protein expression was quantified using the AzureSpot software and normalized to vinculin expression. The differential effects of palbociclib on the sensitive and resistance lines were examined by treating both lines with 500 nM palbocicilib for 24 h and comparing to their relative controls. The effect of elacestrant, fulvestrant and palbociclib in vivo was examined in end of study tumors which were collected 4 h post-last dose.

### Examples

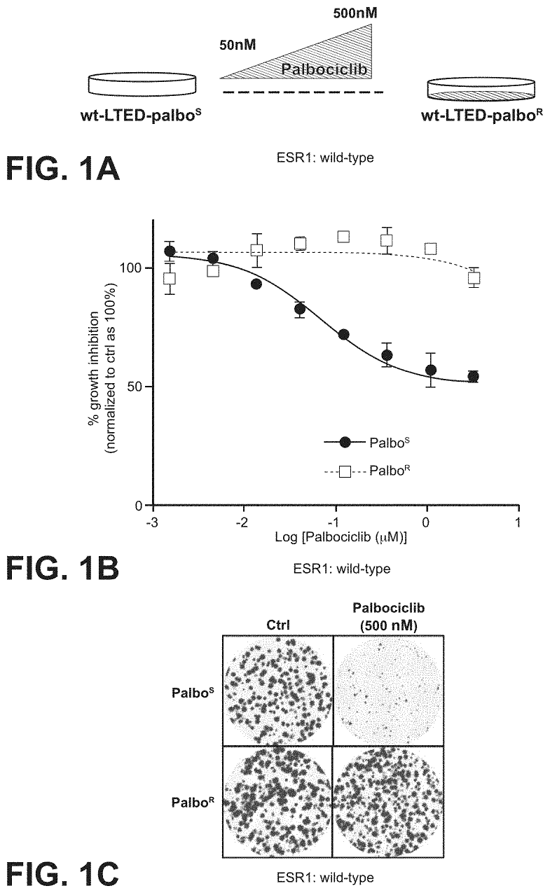

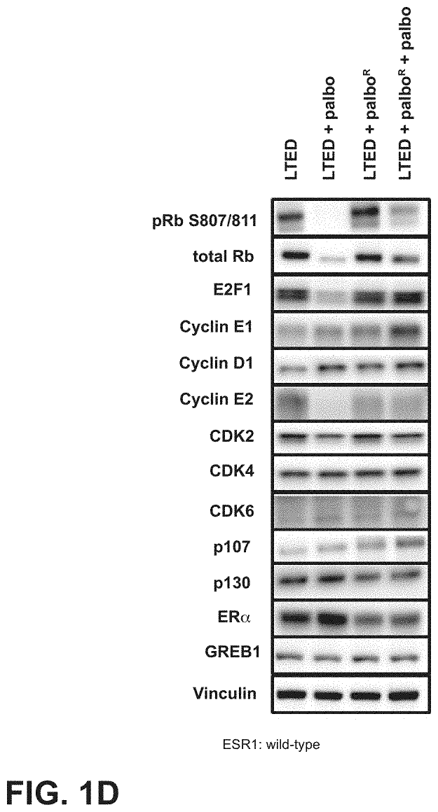

Referring to FIGS. 1A-1D, the generation and characterization of palbociclib-resistant models in wild-type and mutant ESR1 backgrounds is provided. In FIG. 1A, the resistance to palbociclib for the generated ESR1:wild-type LTED cell lines is plotted for the palbociclib sensitive (palbo^{S}) and palbociclib resistant (palbo^{R}) cell lines. In FIG. 1B, the percent growth inhibition (normalized to control as 100%) with respect to the Log[Palbociclib(μM)] is plotted for the palbo^{S} and palbo^{R} ESR1:wild-type cell lines. In FIG. 1C, colony forming assay pictures of the palbo^{S} and palbo^{R} ESR1:wild-type cell lines are provided for the control and after treatment with palbociclib (500 nM). In FIG. 1D, a Western blot is illustrated for the LTED, LTED+palbo, LTED− palbo^{R}, and LTED− palbo^{R}+palbo models having an ESR1:wild-type gene.

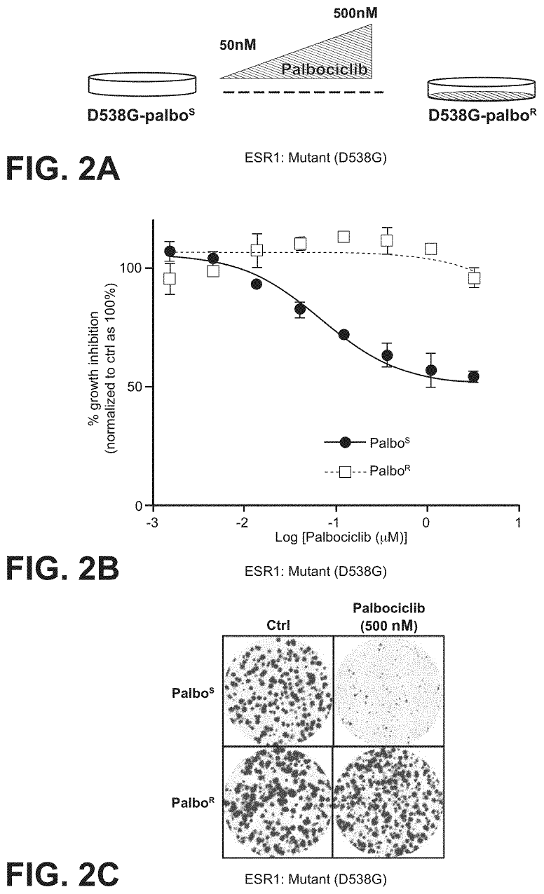

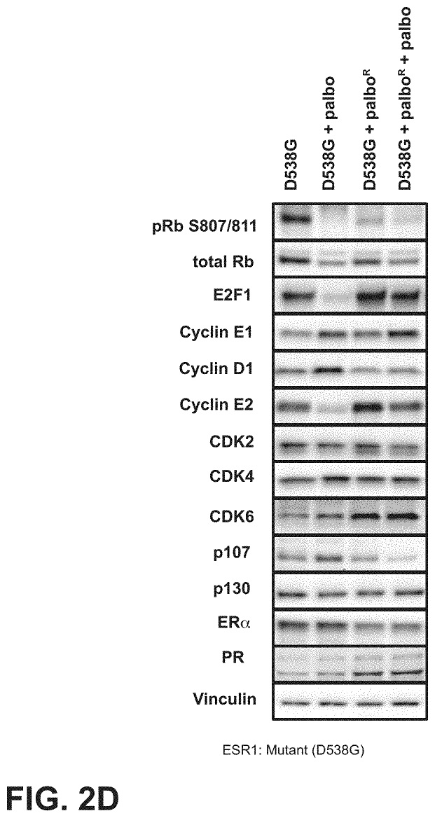

Referring to FIGS. 2A-2D, the generation and characterization of palbociclib-resistant models in wild-type and mutant ESR1 backgrounds is provided. In FIG. 2A, the resistance to palbociclib for the generated ESR1:D538G LTED cell lines is plotted for the palbociclib sensitive (palbo^{S}) and palbociclib resistant (palbo^{R}) cell lines. In FIG. 2B, the percent growth inhibition (normalized to control as 100%) with respect to the Log[Palbociclib(μM)] is plotted for the palbo^{S} and palbo^{R} ESR1:D538G cell lines. In FIG. 2C, colony forming assay pictures of the palbo^{S} and palbo^{R} ESR1:D538G cell lines are provided for the control and after treatment with palbociclib (500 nM). In FIG. 2D, a Western blot is illustrated for the LTED, LTED+palbo, LTED− palbo^{R}, and LTED− palbo^{R}+palbo models having an ESR1:D538G mutation.

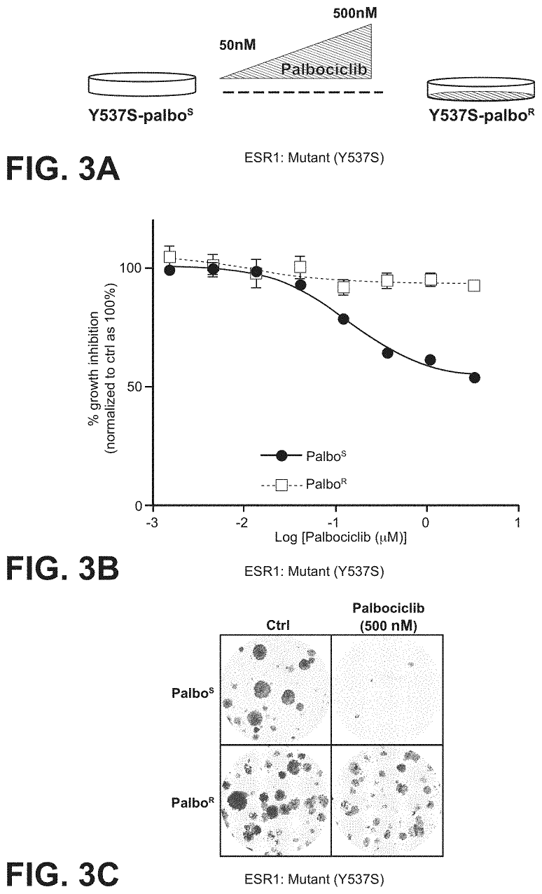

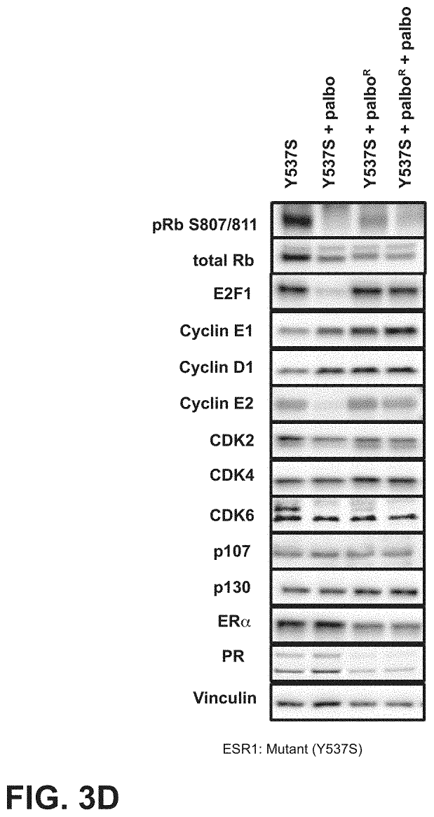

Referring to FIGS. 3A-3D, the generation and characterization of palbociclib-resistant models in wild-type and mutant ESR1 backgrounds is provided. In FIG. 3A, the resistance to palbociclib for the generated ESR1:Y537S LTED cell lines is plotted for the palbociclib sensitive (palbo^{S}) and palbociclib resistant (palbo^{R}) cell lines. In FIG. 3B, the percent growth inhibition (normalized to control as 100%) with respect to the Log[Palbociclib(μM)] is plotted for the palbo^{S} and palbo^{R} ESR1:Y537S cell lines. In FIG. 3C, colony forming assay pictures of the palbo^{S} and palbo^{R} ESR1:Y537S cell lines are provided for the control and after treatment with palbociclib (500 nM). In FIG. 3D, a Western blot is illustrated for the LTED, LTED+palbo, LTED− palbo^{R}, and LTED− palbo^{R}+palbo models having an ESR1:Y537S mutation.

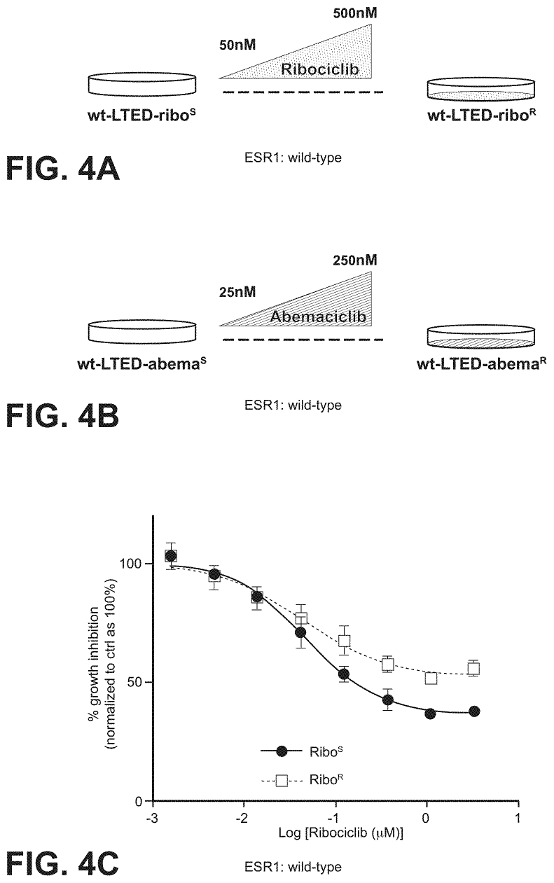

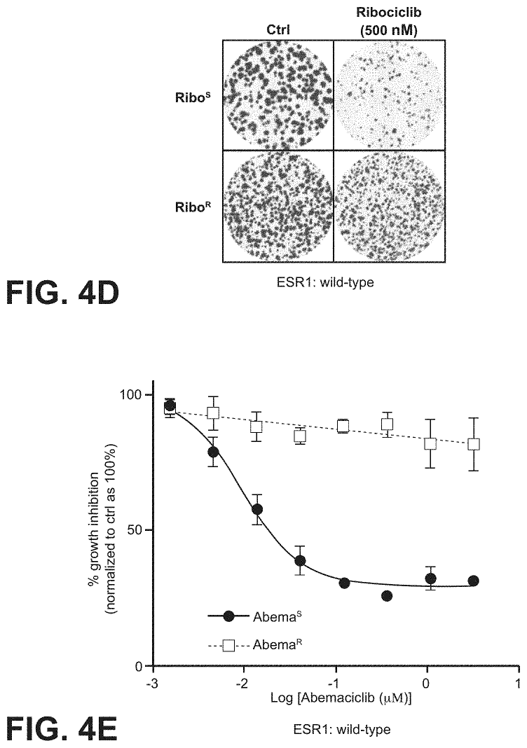

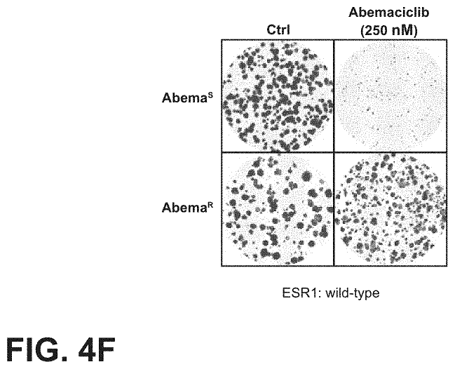

Referring now to FIGS. 4A-4F, the generation and characterization of ribociclib- and abemaciclib-resistant models in wild-type and mutant ESR1 backgrounds is provided. In FIG. 4A, the resistance to ribociclib for the generated ESR1:wild-type LTED cell lines is plotted for the ribociclib sensitive (ribo^{S}) and ribociclib resistant (ribo^{R}) cell lines. In FIG. 4B, the resistance to abemaciclib for the generated ESR1:wild-type LTED cell lines is plotted for the abemaciclib sensitive (abema^{S}) and abemaciclib resistant (abema^{R}) cell lines. In FIG. 4C, the percent growth inhibition (normalized to control as 100%) with respect to the Log[Ribociclib(μM)] is plotted for the ribo^{S} and ribo^{R} ESR1:wild-type cell lines. In FIG. 4D, colony forming assay pictures of the ribo^{S} and ribo^{R} ESR1:wild-type cell lines are provided for the control and after treatment with ribociclib (500 nM). In FIG. 4E, the percent growth inhibition (normalized to control as 100%) with respect to the Log[Abemaciclib(μM)] is plotted for the abema^{S} and abema^{R} ESR1:wild-type cell lines. In FIG. 4F, colony forming assay pictures of the abema^{S} and abema^{R} ESR1:wild-type cell lines are provided for the control and after treatment with abemaciclib (500 nM).

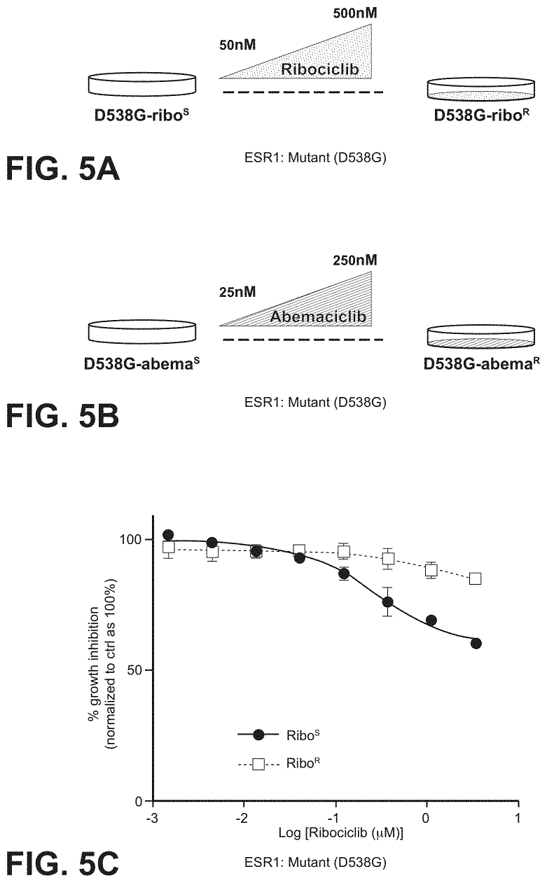

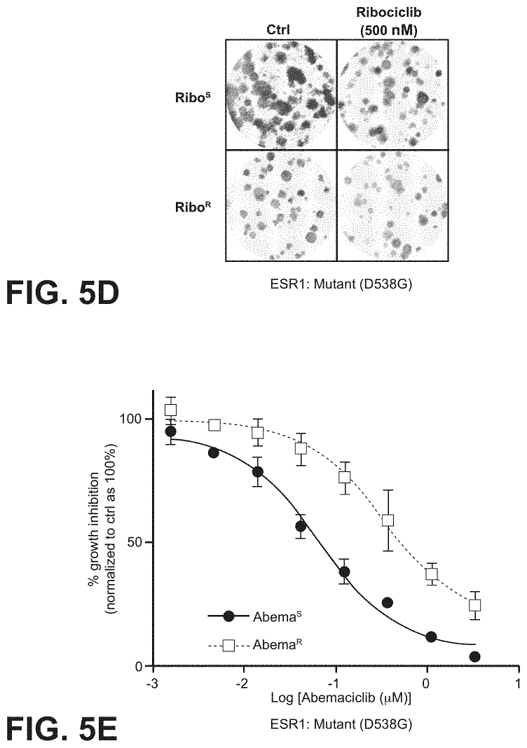

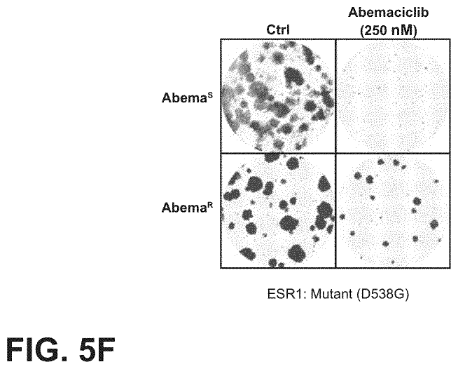

Referring now to FIGS. 5A-5F, the generation and characterization of ribociclib- and abemaciclib-resistant models in wild-type and mutant ESR1 backgrounds is provided. In FIG. 5A, the resistance to ribociclib for the generated ESR1:D538G LTED cell lines is plotted for the ribociclib sensitive (ribo^{S}) and ribociclib resistant (ribo^{R}) cell lines. In FIG. 5B, the resistance to abemaciclib for the generated ESR1:D538G LTED cell lines is plotted for the abemaciclib sensitive (abema^{S}) and abemaciclib resistant (abema^{R}) cell lines. In FIG. 5C, the percent growth inhibition (normalized to control as 100%) with respect to the Log[Ribociclib(μM)] is plotted for the ribo^{S} and ribo^{R} ESR1:D538G cell lines. In FIG. 5D, colony forming assay pictures of the ribo^{S} and ribo^{R} ESR1:D538G cell lines are provided for the control and after treatment with ribociclib (500 nM). In FIG. 5E, the percent growth inhibition (normalized to control as 100%) with respect to the Log[Abemaciclib(μM)] is plotted for the abema^{S} and abema^{R} ESR1:D538G cell lines. In FIG. 5F, colony forming assay pictures of the abema^{S} and abema^{R} ESR1:D538G cell lines are provided for the control and after treatment with abemaciclib (500 nM).

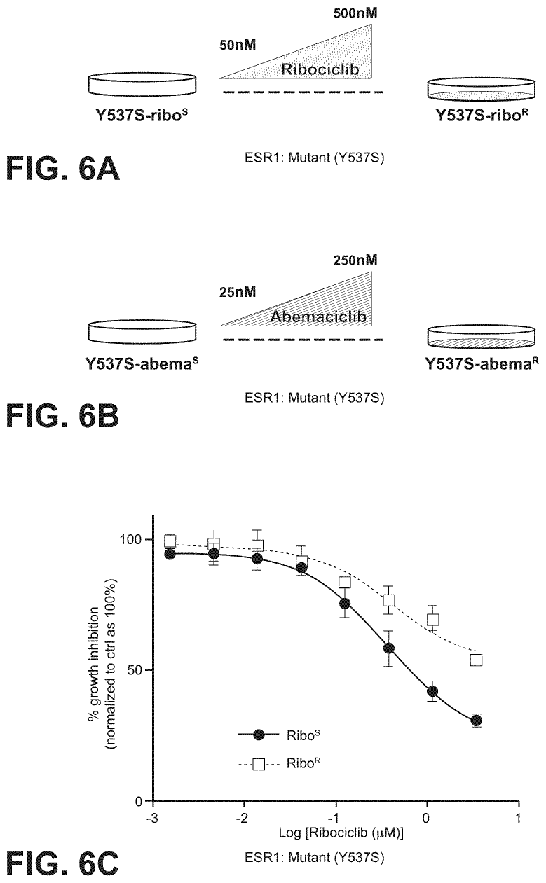

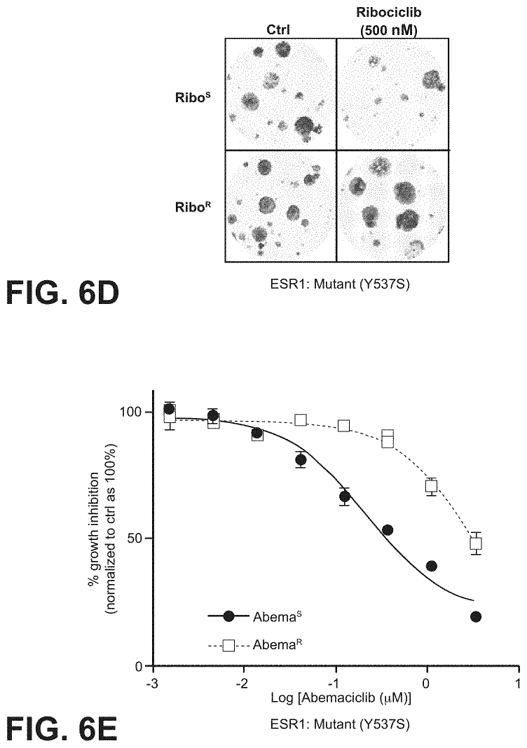

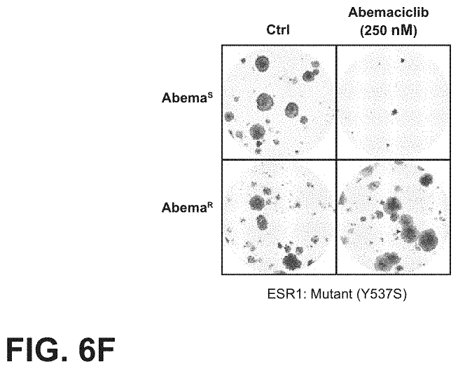

Referring now to FIGS. 6A-6F, the generation and characterization of ribociclib- and abemaciclib-resistant models in wild-type and mutant ESR1 backgrounds is provided. In FIG. 6A, the resistance to ribociclib for the generated ESR1:Y537S LTED cell lines is plotted for the ribociclib sensitive (ribo^{S}) and ribociclib resistant (ribo^{R}) cell lines. In FIG. 6B, the resistance to abemaciclib for the generated ESR1:Y537S LTED cell lines is plotted for the abemaciclib sensitive (abema^{S}) and abemaciclib resistant (abema^{R}) cell lines. In FIG. 6C, the percent growth inhibition (normalized to control as 100%) with respect to the Log[Ribociclib(μM)] is plotted for the ribo^{S} and ribo^{R} ESR1:Y537S cell lines. In FIG. 6D, colony forming assay pictures of the ribo^{S} and ribo^{R} ESR1:Y537S cell lines are provided for the control and after treatment with ribociclib (500 nM). In FIG. 6E, the percent growth inhibition (normalized to control as 100%) with respect to the Log[Abemaciclib(μM)] is plotted for the abema^{S} and abema^{R} ESR1:Y537S cell lines. In FIG. 6F, colony forming assay pictures of the abema^{S} and abema^{R} ESR1:Y537S cell lines are provided for the control and after treatment with abemaciclib (500 nM).

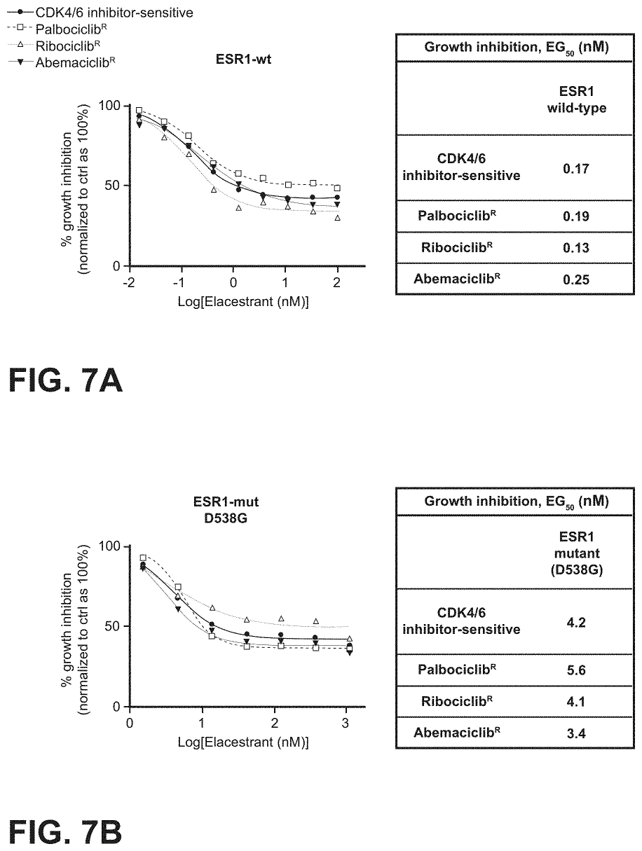

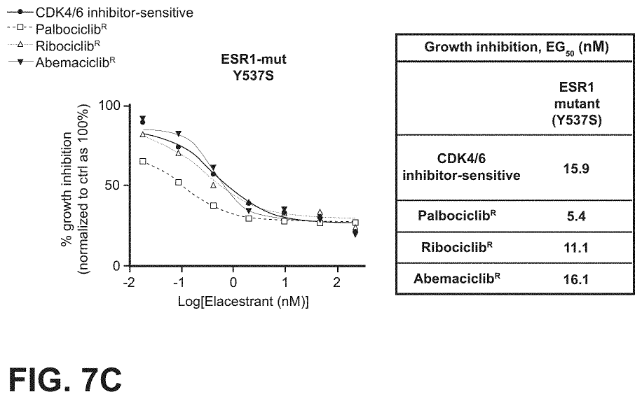

Referring now to FIGS. 7A-7C, elacestrant demonstrated dose-dependent inhibition of tumor growth and tumor regression regardless of prior treatment history or ESR1 status. In FIG. 7A, the EC₅₀ (nM) values are provided and the percent growth inhibition is plotted with respect to the Log[Elacestrant (nM)] for the ESR1:wild-type CDK4/6 inhibitor sensitive, ESR1:wild-type palbociclib^{R}, ESR1:wild-type ribociclib^{R}, and ESR1:wild-type abemaciclib^{R} cell lines. In FIG. 7B, the EC₅₀ (nM) values are provided and the percent growth inhibition is plotted with respect to the Log[Elacestrant (nM)] for the ESR1:D538G CDK4/6 inhibitor sensitive, ESR1:D538G palbociclib^{R}, ESR1:D538G ribociclib^{R}, and ESR1:D538G abemaciclib^{R} cell lines. In FIG. 7C, the EC₅₀ (nM) values are provided and the percent growth inhibition is plotted with respect to the Log[Elacestrant (nM)] for the ESR1:Y537S CDK4/6 inhibitor sensitive, ESR1:Y537S palbociclib^{R}, ESR1:Y537S ribociclib^{R}, and ESR1:Y537S abemaciclib^{R} cell lines.

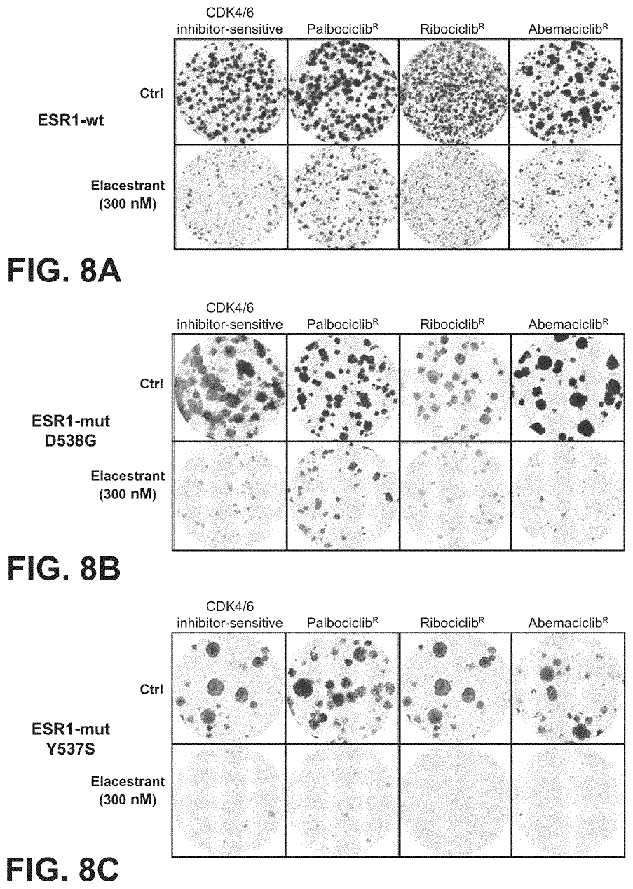

Referring to FIGS. 8A-8C, elacestrant demonstrated long-term growth inhibition in CDK4/6 inhibitor-resistant cell lines. In FIG. 8A, the colony forming assay pictures in the top row visualize the growth for the control ESR1:wild-type CDK4/6 inhibitor sensitive, ESR1:wild-type palbociclib^{R}, ESR1:wild-type ribociclib^{R}, and ESR1:wild-type abemaciclib^{R} cell lines while the pictures in the bottom row visualize the cell growth for the control ESR1:wild-type CDK4/6 inhibitor sensitive, ESR1:wild-type palbociclib^{R}, ESR1:wild-type ribociclib^{R}, and ESR1:wild-type abemaciclib^{R} cell lines after being treated with elacestrant (300 nM). In FIG. 8B, the colony forming assay pictures in the top row visualize the growth for the control ESR1:D538G CDK4/6 inhibitor sensitive, ESR1:D538G palbociclib^{R}, ESR1:D538G ribociclib^{R}, and ESR1:D538G abemaciclib^{R} cell lines while the pictures in the bottom row visualize the cell growth for the control ESR1:D538G CDK4/6 inhibitor sensitive, ESR1:D538G palbociclib^{R}, ESR1:D538G ribociclib^{R}, and ESR1:D538G abemaciclib^{R} cell lines after being treated with elacestrant (300 nM). In FIG. 8C, the colony forming assay pictures in the top row visualize the growth for the control ESR1:Y537S CDK4/6 inhibitor sensitive, ESR1:Y537S palbociclib^{R}, ESR1:Y537A ribociclib^{R}, and ESR1:Y537S abemaciclib^{R} cell lines while the pictures in the bottom row visualize the cell growth for the control ESR1:Y537S CDK4/6 inhibitor sensitive, ESR1:Y537S palbociclib^{R}, ESR1:Y537S ribociclib^{R}, and ESR1:Y537S abemaciclib^{R} cell lines after being treated with elacestrant (300 nM).

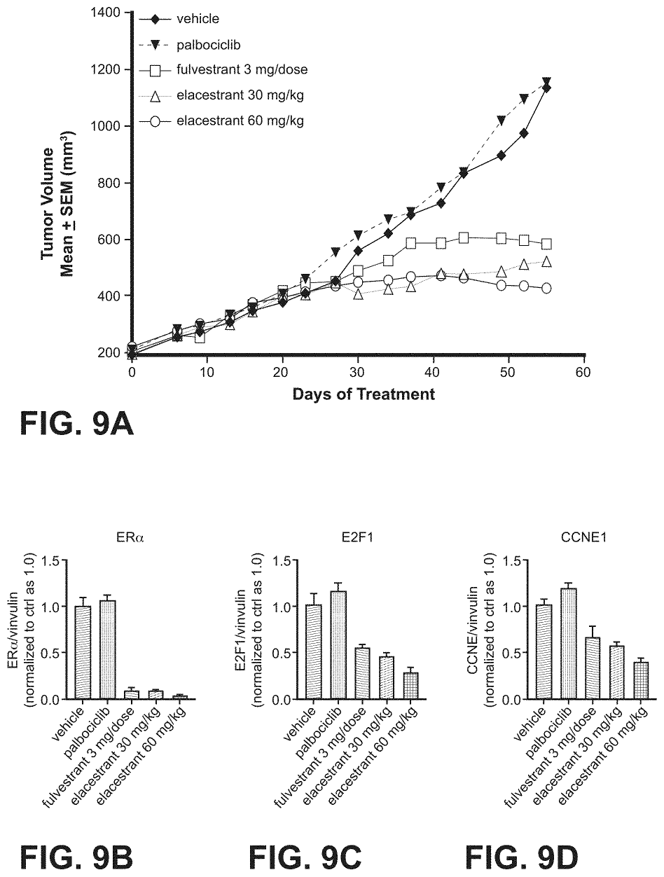

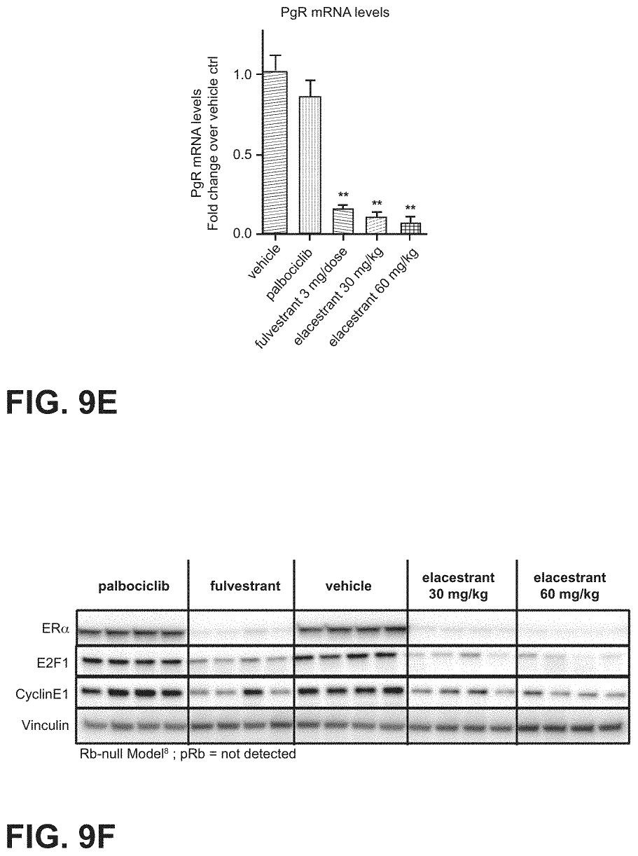

Referring now to FIGS. 9A-9F, elacestrant demonstrated inhibited growth of a PDX model insensitive to palbociclib. In FIG. 9A, average tumor volumes over time in athymic nude mice implanted with the WHIM43 PDX xenograft with an ESR1:D538G mutation were treated with a vehicle control, palbociclib, fulvestrant (3 mg/dose), and elacestrant (30 and 60 mg/kg). In FIG. 9B, the quantification of ERα protein level was determined by plotting ERα/vinculin (normalized to control) against treatment of the WHIM43 PDX xenograft model having an ESR1:D538G mutation with a vehicle control, palbociclib, fulvestrant (3 mg/dose), and elacestrant (30 and 60 mg/kg). In FIG. 9C, the quantification of E2F1 protein level was determined by plotting E2F1/vinculin (normalized to control) against treatment of the WHIM43 PDX xenograft model having an ESR1:D538G mutation with a vehicle control, palbociclib, fulvestrant (3 mg/dose), and elacestrant (30 and 60 mg/kg). In FIG. 9D, the quantification of CCNE1 protein (or Cyclin E1) level was determined by plotting PgR mRNA levels (normalized to control) against treatment of the WHIM43 PDX xenograft model having an ESR1:D538G mutation with a vehicle control, palbociclib, fulvestrant (3 mg/dose), and elacestrant (30 and 60 mg/kg). In FIG. 9E, the PgR mRNA levels were determined by plotting PgR mRNA levels (normalized to control) against treatment of the WHIM43 PDX xenograft model having an ESR1:D538G mutation with a vehicle control, palbociclib, fulvestrant (3 mg/dose), and elacestrant (30 and 60 mg/kg). In FIG. 9F, a Western blot illustrating the WHIM43 PDX xenograft model having an ESR1:D538G mutation treated with a vehicle control, palbociclib, fulvestrant (3 mg/dose), and elacestrant (30 and 60 mg/kg) is provided.

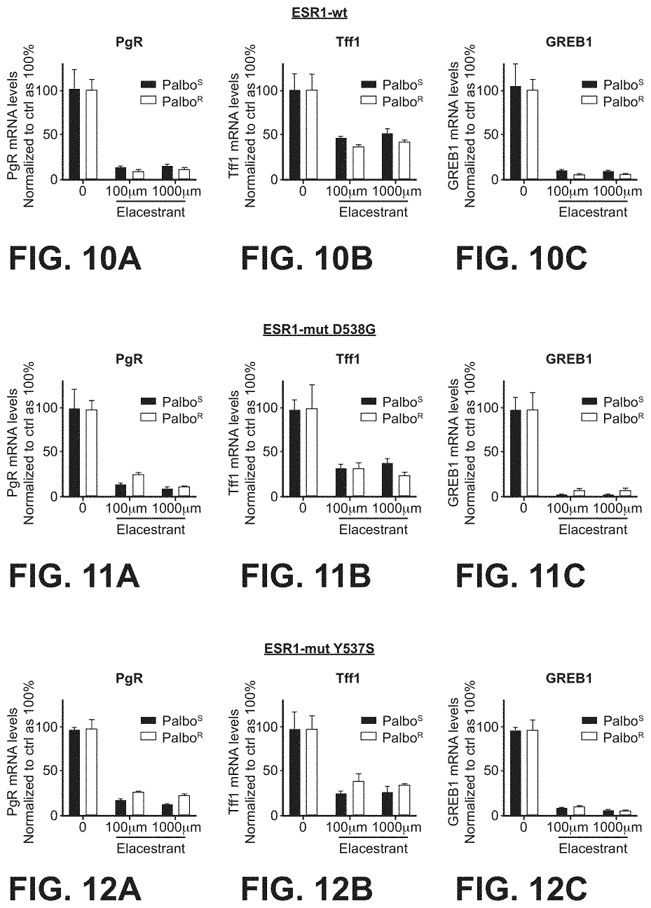

Referring now to FIGS. 10A-10C, elacestrant is demonstrated to inhibit ER signaling in CDK4/6 inhibitor-resistant models. In FIG. 10A, the quantification of progesterone receptor (PgR) in tumor cell models having a an ESR1:wild-type mutation was determined by plotting PgR mRNA levels (normalized to control) against treatment of the palbo^{S} and palbo^{R} cell lines with control or elacestrant (100 nM and 1000 nM). In FIG. 10B, the quantification of trefoil factor 1 (TFF1) in tumor cell models having an ESR1:wild-type mutation was determined by plotting TFF1 mRNA levels (normalized to control) against treatment of the palbo^{S} and palbo^{R} cell lines with control or elacestrant (100 nM and 1000 nM). In FIG. 10C, the quantification of growth regulated by estrogen (GREB1) in tumor cell models having an ESR1:wild-type mutation was determined by plotting GREB1 mRNA levels (normalized to control) against treatment of the palbo^{S} and palbo^{R} cell lines with control or elacestrant (100 nM and 1000 nM).

Referring now to FIGS. 11A-11C, elacestrant is demonstrated to inhibit ER signaling in CDK4/6 inhibitor-resistant models. In FIG. 11A, the quantification of progesterone receptor (PgR) in tumor cell models having a an ESR1:D538G mutation was determined by plotting PgR mRNA levels (normalized to control) against treatment of the palbo^{S} and palbo^{R} cell lines with control or elacestrant (100 nM and 1000 nM). In FIG. 11B, the quantification of trefoil factor 1 (TFF1) in tumor cell models having an ESR1:D538G mutation was determined by plotting TFF1 mRNA levels (normalized to control) against treatment of the palbo^{S} and palbo^{R} cell lines with control or elacestrant (100 nM and 1000 nM). In FIG. 11C, the quantification of growth regulated by estrogen (GREB1) in tumor cell models having an ESR1:D538G mutation was determined by plotting GREB1 mRNA levels (normalized to control) against treatment of the palbo^{S} and palbo^{R} cell lines with control or elacestrant (100 nM and 1000 nM).

Referring now to FIGS. 12A-12C, elacestrant is demonstrated to inhibit ER signaling in CDK4/6 inhibitor-resistant models. In FIG. 12A, the quantification of progesterone receptor (PgR) in tumor cell models having a an ESR1:Y537S mutation was determined by plotting PgR mRNA levels (normalized to control) against treatment of the palbo^{S} and palbo^{R} cell lines with control or elacestrant (100 nM and 1000 nM). In FIG. 11B, the quantification of trefoil factor 1 (TFF1) in tumor cell models having an ESR1:Y537S mutation was determined by plotting TFF1 mRNA levels (normalized to control) against treatment of the palbo^{S} and palbo^{R} cell lines with control or elacestrant (100 nM and 1000 nM). In FIG. 11C, the quantification of growth regulated by estrogen (GREB1) in tumor cell models having an ESR1:Y537S mutation was determined by plotting GREB1 mRNA levels (normalized to control) against treatment of the palbo^{S} and palbo^{R} cell lines with control or elacestrant (100 nM and 1000 nM).

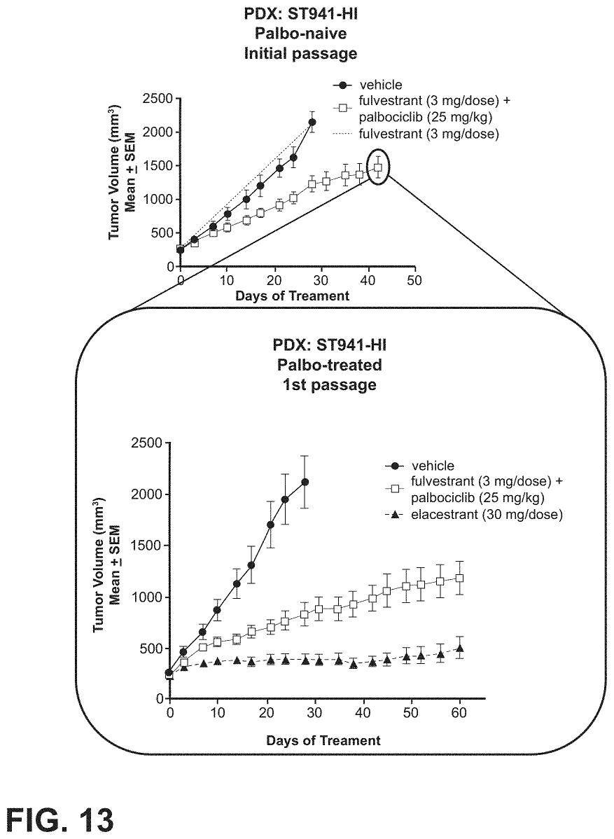

Referring now to FIG. 13, elacestrant is demonstrated to inhibit tumor growth of a PDX model previously treated with a combination of fulvestrant and palbociclib. In FIG. 13, the tumor volume of the ST941-HI PDX xenograft model (treatment-naïve) is plotted against the days of treatment, where the model was treated with vehicle or a combination of fulvestrant and palbociclib (the fulvestrant 3 mg/dose data is from a separate study). The tumors from the fulvestrant and palbociclib arm were then re-implanted into another study (ST941-HI Palbociclib-treated; first passage) and then subsequently treated with the vehicle, fulvestrant (3 mg/dose), palbociclib (25 mg/kg), and elacestrant (30 mg/kg) to demonstrate the elacestrant is still effective at inhibiting tumor growth in the PDX model previously treated with a combination of fulvestrant and palbociclib.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method of inhibiting and degrading a CDK4/6 inhibitor resistant estrogen receptor alpha positive cancer in a subject comprising administering to the subject a therapeutically effective amount of elacestrant, or a pharmaceutically acceptable salt or solvate thereof.

2. The method of claim 1, wherein the CDK4/6 inhibitor resistant estrogen receptor alpha positive cancer is resistant to palbociclib, ribociclib, abemaciclib, or a combination thereof.

3. The method of claim 1, wherein the CDK4/6 inhibitor resistant estrogen receptor alpha positive cancer is resistant to palbociclib.

4. The method of claim 1, wherein the CDK4/6 inhibitor resistant estrogen receptor alpha positive cancer is resistant to ribociclib.

5. The method of claim 1, wherein the CDK4/6 inhibitor resistant estrogen receptor alpha positive cancer is resistant to abemaciclib.

6. The method of any one of claims 1-5, wherein the CDK4/6 inhibitor resistant estrogen receptor alpha positive cancer comprises one or more mutations selected from the group consisting of D538G, Y537X₁, L536X₂, P535H, V534E, S463P, V392I, E380Q and combinations thereof, wherein: X₁ is S, N, or C; and X₂ is R or Q.

7. The method of claim 6, wherein the mutation is Y537S.

8. The method of claim 6, wherein the mutation is D538G.

9. The method of any one of claims 1-8, wherein the CDK4/6 inhibitor resistant estrogen receptor alpha positive cancer is additionally resistant to a drug selected from the group consisting of anti-estrogens, aromatase inhibitors, and combinations thereof.

10. The method of any one of claims 1-9, wherein the CDK4/6 inhibitor resistant estrogen receptor alpha positive cancer is selected from the group consisting of breast cancer, uterine cancer, ovarian cancer, and pituitary cancer.

11. The method of any one of claims 1-10, wherein the CDK4/6 inhibitor resistant estrogen receptor alpha positive cancer is advanced or metastatic breast cancer.

12. The method of any one of claims 1-10, wherein the CDK4/6 inhibitor resistant estrogen receptor alpha positive cancer is breast cancer.

13. The method of any one of claims 1-12, wherein the subject is a post-menopausal woman.

14. The method of any one of claims 1-12, wherein the subject is a pre-menopausal woman.

15. The method of any one of claims 1-12, wherein the subject is a post-menopausal woman who had relapsed or progressed after previous treatment with selective estrogen receptor modulators (SERMs) and/or aromatase inhibitors (AIs).

16. The method of any one of claims 1-15, wherein the elacestrant is administered to the subject at a dose of from about 200 mg/day to about 500 mg/day.

17. The method of any one of claims 1-16, wherein the elacestrant is administered to the subject at a dose of about 200 mg/day, about 300 mg/day, about 400 mg/day, or about 500 mg/day.

18. The method of any one of claims 1-15, wherein the elacestrant is administered to the subject at a dose that is the maximum tolerated dose for the subject.

19. The method of any one of claims 1-18, the method further comprising:
identifying the subject for treatment by measuring increased expression of one or more genes selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL.

20. The method according to claim 19, wherein the one or more genes is selected from AKT1, AKT2, BRAF, CDK4, CDK6, PIK3CA, PIK3R1, and MTOR.

21. The method of any one of claims 1-20, wherein the ratio of the concentration of elacestrant or a salt or solvate thereof in the tumor to the concentration of elacestrant or a salt or solvate thereof in plasma (T/P) following administration is at least about 15.

22. A method of treating a CDK4/6 inhibitor resistant estrogen receptor alpha-positive cancer in a subject having a wild-type estrogen receptor alpha and/or a mutant estrogen receptor alpha, the method comprising:
administering to the subject a therapeutically effective amount of elacestrant, or a pharmaceutically acceptable salt or solvate thereof,
wherein the mutant estrogen receptor alpha comprises one or more mutations selected from the group consisting of D538G, Y537X₁, L536X₂, P535H, V534E, S463P, V392I, E380Q and combinations thereof, wherein: X₁ is S, N, or C; and X₂ is R or Q.

23. The method of claim 22, wherein the CDK4/6 inhibitor resistant estrogen receptor alpha positive cancer is resistant to palbociclib, ribociclib, abemaciclib, or a combination thereof.

24. The method of claim 22, wherein the CDK4/6 inhibitor resistant estrogen receptor alpha positive cancer is resistant to palbociclib.

25. The method of claim 22, wherein the CDK4/6 inhibitor resistant estrogen receptor alpha positive cancer is resistant to ribociclib.

26. The method of claim 22, wherein the CDK4/6 inhibitor resistant estrogen receptor alpha positive cancer is resistant to abemaciclib.

27. The method of any one of claims 22-26, wherein the CDK4/6 inhibitor resistant estrogen receptor alpha positive cancer is additionally resistant to a drug selected from the group consisting of anti-estrogens, aromatase inhibitors, and combinations thereof.

28. The method of claim 27, wherein the anti-estrogens are selected from the group consisting of tamoxifen, toremifene and fulvestrant and the aromatase inhibitors are selected from the group consisting of exemestane, letrozole and anastrozole.

29. The method of any one of claims 22-28, wherein the CDK4/6 inhibitor resistant estrogen receptor alpha-positive cancer is selected from the group consisting of breast cancer, uterine cancer, ovarian cancer, and pituitary cancer.

30. The method of any one of claims 22-29, wherein the CDK4/6 inhibitor resistant estrogen receptor alpha positive cancer is advanced or metastatic breast cancer.

31. The method of any one of claims 22-29, wherein the CDK4/6 inhibitor resistant estrogen receptor alpha positive cancer is breast cancer.

32. The method of any one of claims 22-31, wherein the subject is a post-menopausal woman.

33. The method of any one of claims 22-31, wherein the subject is a pre-menopausal woman.

34. The method of any one of claims 22-31, wherein the subject is a post-menopausal woman who had relapsed or progressed after previous treatment with SERMs and/or AIs.

35. The method of any one of claims 22-34, wherein said subject expresses at least one mutant estrogen receptor alpha selected from the group consisting of D538G, Y537S, Y537N, Y537C, E380Q, S463P, L536R, L536Q, P535H, V392I and V534E.

36. The method of claim 35, wherein the mutation includes Y537S.

37. The method of claim 35, wherein the mutation includes D538G.

38. The method of any one of claims 22-37, the method further comprising:
identifying the subject for treatment by measuring increased expression of one or more genes selected from ABL1, AKT1, AKT2, ALK, APC, AR, ARID1A, ASXL1, ATM, AURKA, BAP, BAP1, BCL2L11, BCR, BRAF, BRCA1, BRCA2, CCND1, CCND2, CCND3, CCNE1, CDH1, CDK4, CDK6, CDK8, CDKN1A, CDKN1B, CDKN2A, CDKN2B, CEBPA, CTNNB1, DDR2, DNMT3A, E2F3, EGFR, EML4, EPHB2, ERBB2, ERBB3, ESR1, EWSR1, FBXW7, FGF4, FGFR1, FGFR2, FGFR3, FLT3, FRS2, HIF1A, HRAS, IDH1, IDH2, IGF1R, JAK2, KDM6A, KDR, KIF5B, KIT, KRAS, LRP1B, MAP2K1, MAP2K4, MCL1, MDM2, MDM4, MET, MGMT, MLL, MPL, MSH6, MTOR, MYC, NF1, NF2, NKX2-1, NOTCH1, NPM, NRAS, PDGFRA, PIK3CA, PIK3R1, PML, PTEN, PTPRD, RARA, RB1, RET, RICTOR, ROS1, RPTOR, RUNX1, SMAD4, SMARCA4, SOX2, STK11, TET2, TP53, TSC1, TSC2, and VHL.

39. The method according to claim 38, wherein the one or more genes is selected from AKT1, AKT2, BRAF, CDK4, CDK6, PIK3CA, PIK3R1, and MTOR.

40. The method of any one of claims 22-39, wherein the elacestrant is administered to the subject at a dose of from about 200 to about 500 mg/day.

41. The method of any one of claims 22-40, wherein the elacestrant is administered to the subject at a dose of about 200 mg, about 300 mg, about 400 mg, or about 500 mg.

42. The method of any one of claims 22-41, wherein the ratio of the concentration of elacestrant or a salt or solvate thereof in the tumor to the concentration of elacestrant or a salt or solvate thereof in plasma (T/P) following administration is at least about 15.
